SEQUENCE LISTING (2 sequences)

SEQ ID NO: 1 - RNA, 15 nt, synthetic construct
  source 1..15: mol_type = other RNA
aaaaaaaaaaaaaaa

SEQ ID NO: 2 - DNA, 10 bp, synthetic construct
  source 1..10: mol_type = other DNA
aaaaaaaaaa

(19) United States
(12) Patent Application Publication
(10) Pub. No.: US 2026/0132449 A1
(43) Pub. Date: May 14, 2026

(54) METHODS, COMPOSITIONS, AND KITS FOR DETERMINING THE LOCATION OF MULTIPLE ANALYTES IN A BIOLOGICAL SAMPLE

(71) Applicant: 10x Genomics, Inc., Pleasanton, CA (US)

(72) Inventors: Christina Galonska, Stockholm (SE); Malte Kuhnemund, Stockholm (SE)

(21) Appl. No.: 19/447,151

(22) Filed: Jan. 13, 2026

Related U.S. Application Data
(63) Continuation of application No. 18/914,833, filed on Oct. 14, 2024, now Pat. No. 12,571,029.
Application No. 18/914,833 is a continuation of application No. PCT/US2023/079071, filed on Nov. 8, 2023.
(60) Provisional application No. 63/424,056, filed on Nov. 9, 2022.

(51) Int. Cl. C12Q 1/6841; C12Q 1/682

(52) U.S. Cl. CPC C12Q 1/6841; C12Q 1/682

(57) ABSTRACT

Provided herein are methods, compositions, and kits for the spatial analysis of target nucleic acids, or complements thereof, by their 5′ end.

### CROSS-REFERENCE TO RELATED APPLICATIONS

This application is a continuation of U.S. patent application Ser. No. 18/914,833, filed Oct. 14, 2024, which is a continuation of International Application PCT/US2023/079071, with an international filing date of Nov. 8, 2023, which claims the benefit of priority to U.S. Provisional Patent Application No. 63/424,056, filed on Nov. 9, 2022, the contents of which are incorporated herein by in its entirety.

### SEQUENCE LISTING

This application contains a Sequence Listing that has been submitted electronically as an XML file named “47706-0333002_ST26_SL.XML.” The XML file, created on Jan. 8, 2026, is 2,842 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small number of analytes in the context of an intact tissue or a portion of a tissue, or provide substantial analyte data for dissociated tissue (e.g., single cells), but fail to provide information regarding the position of the single cell in a parent biological sample (e.g., tissue sample).

Sequencing nucleic acid libraries generated from spatial array analysis generally biases capture to sequences from the 3′ end of the captured analytes. However, 5′ end sequences can contain valuable information about the target analyte. Thus, strategies are needed to sequence regions more than about 1 kilobase away from the 3′ end of analytes, such as 5′ ends, in nucleic acid libraries generated from spatial array analysis. Additionally, combining techniques to sequence regions more than about 1 kilobase away from the 3′ end of analytes with other capture techniques allows for multiplex capture of analytes from a biological sample.

### SUMMARY

The present disclosure features methods of capturing analytes on a spatial array, where the spatial array includes a plurality of capture probes and a capture probe of the plurality of capture probes includes a spatial barcode and a capture domain. Since capture on a spatial array can be generally biased toward the capture 3′ end sequences of nucleic acid analytes, methods are still needed to capture the 5′ ends of the nucleic acid analytes or complements thereof. For example, target RNA nucleic acid analytes can be reverse transcribed with a primer including a sequence complementary to a target nucleic acid and a functional domain (e.g., a sequencing adapter) to generate an RNA/DNA (e.g., cDNA duplex). The reverse transcriptase can incorporate a non-templated polynucleotide sequence to the end of the cDNA that hybridizes to the capture domain of the capture probe (e.g., a second capture probe). The target nucleic acid can also be digested resulting in a single-stranded cDNA product that is captured by a capture probe on the array. In addition, such methods for capturing the 5′ end of nucleic acid analytes can be combined with additional capture techniques that capture nucleic acids that serve as a proxy for a nucleic acid analyte. For example, pairs of probes specific to a target nucleic acid can hybridize to a nucleic acid target and subsequently be ligated (e.g., nucleic acid templated ligation). At least one probe of the pair of probes can include a nucleic acid sequence that hybridizes to the capture domain of a capture probe (e.g., a first capture probe). Thus, a proxy of a target nucleic acid can be captured (e.g., to permit general gene expression analysis) in conjunction with capture products that permit analysis of the 5′ proximal sequences of target nucleic acids (e.g., encoding for immune cell receptors).

Thus, provided herein are methods for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a first primer including a sequence that hybridizes to a complementary sequence in the second target nucleic acid, and a functional domain; (b) hybridizing the first probe and the second probe to the first target nucleic acid; (c) generating a ligation product by ligating the first probe to the second probe; (d) hybridizing the first capture sequence of the ligation product to the first capture domain on an array, where the array includes a first plurality of capture probes, where a first capture probe of the first plurality of capture probes includes: a first spatial barcode and the first capture domain; (e) hybridizing the first primer to the second target nucleic acid and extending the first primer using the second target nucleic acid as a template to generate an extension product; (f) incorporating a polynucleotide sequence including at least three nucleotides to the 3′ end of the extension product; (g) hybridizing a second primer to the polynucleotide sequence of the extension product of step (f), where the second primer includes a second capture sequence; (h) extending the extension product of step (g) using the second primer as a template, thereby incorporating a complement of the second capture sequence into the extension product; (i) hybridizing the complement of the second capture sequence of the extension product of (h) to a second capture domain on the array, where the array includes a second plurality of capture probes, where a second capture probe of the second plurality of capture probes includes: a second spatial barcode and the second capture domain; (j) determining (i) the sequence of the first spatial barcode, or a complement thereof, and (ii) all or a portion of the sequence of the ligation product (corresponding to a sequence of the first target nucleic acid) hybridized to the first capture domain, or a complement thereof, and using the determined sequences of (i) and (ii) to determine the location of the first target nucleic acid in the biological sample; and (k) determining (iii) the sequence of the second spatial barcode, or a complement thereof, and (iv) all or a portion of the sequence of the extension product corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first plurality and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate, e.g., the array on the second substrate.

In some embodiments, the hybridizing in steps (d) and/or (i) include passive migration of the ligation product and/or the extension product from the biological sample to the array. In some embodiments, the hybridizing in steps (d) and/or (i) include active migration of the ligation product and/or the extension product from the biological sample to the array, and optionally, where the active migration includes electrophoresis.

In some embodiments, the polynucleotide sequence in step (f) includes a homopolynucleotide sequence, optionally 5′-CCC-3′. In some embodiments, the polynucleotide sequence in step (f) includes a heteropolynucleotide sequence, optionally 5′-CGC-3′.

In some embodiments, the first capture domain includes a first fixed sequence. In some embodiments, the second capture domain includes a second fixed sequence. In some embodiments, the first capture domain and the second capture domain have the same sequence. In some embodiments, the first capture domain and the second capture domain have a different sequence.

In some embodiments, the first primer includes a random sequence. In some embodiments, the random sequence includes a random hexamer. In some embodiments, the random sequence includes a random decamer. In some embodiments, the first primer includes a homopolymer sequence. In some embodiments, the homopolymer sequence includes a poly(T) sequence.

In some embodiments, the first primer includes a sequence substantially complementary to a sequence in the second target nucleic acid encoding a constant region of an antibody. In some embodiments, the first primer includes a sequence substantially complementary to a sequence in the second target nucleic acid encoding a constant region of a B cell receptor. In some embodiments, the first primer includes a sequence substantially complementary to a sequence in the second target nucleic acid encoding a constant region of a T cell receptor. In some embodiments, the functional domain includes a primer binding site or a sequencing specific site.

In some embodiments, the first target nucleic acid is RNA. In some embodiments, the RNA is mRNA. In some embodiments, the first target nucleic acid is DNA. In some embodiments, the second target nucleic acid is RNA. In some embodiments, the RNA is mRNA. In some embodiments, the mRNA includes a sequence encoding a B cell receptor or a fragment thereof. In some embodiments, the mRNA includes a sequence encoding a T cell receptor or a fragment thereof. In some embodiments, the mRNA includes a sequence that does not encode a B cell receptor or a fragment thereof or a T cell receptor or a fragment thereof.

In some embodiments, the extending in step (e) includes the use of a reverse transcriptase.

In some embodiments, incorporating the polynucleotide sequence to the 3′ end of the extension product in step (f) includes the use of a terminal deoxynucleotidyl transferase. In some embodiments, incorporating the polynucleotide sequence to the 3′ end of the extension product in step (f) includes the use of a reverse transcriptase.

In some embodiments, the second primer includes RNA.

In some embodiments, the method includes removing the second target nucleic acid or any other nucleic acid hybridized to the extension product before the complement of the second capture sequence of the extension product hybridizes to the second capture domain of the second capture probe. In some embodiments, the method includes removing the first target nucleic acid, or any other nucleic acid hybridized to the ligation product, before the first capture sequence of the ligation product hybridizes to the first capture domain of the first capture probe. In some embodiments, the removing includes the use of an RNase. In some embodiments, the RNase is RNase H. In some embodiments, the removing includes use of heat.

In some embodiments, the method includes a step of extending the 3′ end of the extension product of step (i) using the second capture probe as a template, thereby generating an extended capture product; and/or extending the second capture probe using the extension product of step (i) as a template, thereby generating a second extended capture probe; and/or a step of extending the 3′ end of the ligation product using the first capture probe as a template, thereby generating an extended ligation product; and/or extending the first capture probe using the ligation product as a template, thereby generating a first extended capture probe.

In some embodiments, the extended capture product is removed from the second capture probe on the array and/or the extended ligation product is removed from the first capture probe on the array. In some embodiments, the removing includes use of heat. In some embodiments, the removing includes use of potassium hydroxide (KOH).

In some embodiments, the first spatial barcode and the second spatial barcode have the same sequence. In some embodiments, the first spatial barcode and the second spatial barcode have a different sequence.

In some embodiments, the determining in step (j) includes sequencing. In some embodiments, the determining in step (k) includes sequencing. In some embodiments, the sequencing includes high-throughput sequencing.

In some embodiments, step (e) is performed after step (c). In some embodiments, step (e) is performed before step (c).

In some embodiments, the first capture sequence and the second capture sequence include the same sequence. In some embodiments, the first capture sequence and the second capture sequence include different sequences.

Also provided herein are methods for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a plurality of primers, where the plurality of primers includes nucleic acid sequences that hybridize to complementary sequences in the second target nucleic acid and a functional domain; (b) hybridizing the first probe and the second probe to the first target nucleic acid; (c) generating a ligation product by ligating the first probe to the second probe; (d) hybridizing the first capture sequence of the ligation product to a first capture domain on an array, where the array includes a first plurality of capture probes, where a first capture probe of the plurality of first capture probes includes: a first spatial barcode and the first capture domain; (e) hybridizing one or more of the plurality of primers to the second target nucleic acid and extending the one or more of the plurality of primers using the second target nucleic acid as a template to generate one or more extension products; (f) incorporating a polynucleotide sequence including at least three nucleotides to the 3′ end of the one or more extension products; (g) hybridizing a second primer to the polynucleotide sequence of the one or more extension products of step (f), where the second primer includes a second capture sequence; (h) extending the one or more extension products of step (g) using the second primer as a template, thereby incorporating a complement of the second capture sequence into the one or more extension products; (i) hybridizing the complement of the second capture sequence of the one or more extension products of (h) to a second capture domain on the array, where the array includes a second plurality of capture probes, where a second capture probe of the second plurality of capture probes includes: a second spatial barcode and the second capture domain; (j) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and for an extension product in the one or more extension products (k) determining (iii) the sequence of the second spatial barcode, or a complement thereof, and (iv) all or a portion of the sequence of one of the one or more extension products corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first plurality and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in steps (d) and/or (i) include passive migration of the ligation product and/or the one or more extension products from the biological sample to the array. In some embodiments, the hybridizing in steps (d) and/or (i) include active migration of the ligation product and/or the one or more extension products from the biological sample to the array, and optionally, where the active migration includes electrophoresis.

In some embodiments, incorporating the polynucleotide sequence in step (f) includes a homopolynucleotide sequence, optionally 5′-CCC-3′. In some embodiments, incorporating the polynucleotide sequence in step (f) includes a heteropolynucleotide sequence, optionally 5′-CGC-3′.

In some embodiments, the first capture domain includes a fixed sequence. In some embodiments, the second capture domain includes a fixed sequence. In some embodiments, the first capture domain and the second capture domain have the same sequence. In some embodiments, the first capture domain and the second capture domain have a different sequence. In some embodiments, one or more primers of the plurality of primers includes a random sequence. In some embodiments, the random sequence includes a random hexamer.

In some embodiments, the random sequence includes a random decamer. In some embodiments, one or more primers of the plurality of primers includes a homopolymer sequence. In some embodiments, the homopolymer sequence includes a poly(T) sequence.

In some embodiments, one or more primers of the plurality of primers includes a sequence substantially complementary to a sequence in the second target nucleic acid encoding a constant region of an antibody. In some embodiments, one or more primers of the plurality of primers includes a sequence substantially complementary to a sequence in the second target nucleic acid encoding a B cell receptor. In some embodiments, one or more primers of the plurality of primers includes a sequence substantially complementary to a sequence in the second target nucleic acid encoding a T cell receptor.

In some embodiments, the functional domain includes a primer binding site or a sequencing specific site.

In some embodiments, the first target nucleic acid is RNA. In some embodiments, the RNA is mRNA. In some embodiments, the first target nucleic acid is DNA. In some embodiments, the second target nucleic acid is RNA. In some embodiments, the RNA is mRNA. In some embodiments, the mRNA includes a sequence encoding a B cell receptor or a fragment thereof. In some embodiments, the mRNA includes a sequence encoding a T cell receptor or a fragment thereof. In some embodiments, the mRNA includes a sequence that does not encode a B cell receptor or a fragment thereof or a T cell receptor or a fragment thereof.

In some embodiments, incorporating the polynucleotide sequence to the 3′ end of the one or more extension products in step (f) includes the use of a terminal deoxynucleotidyl transferase. In some embodiments, incorporating the polynucleotide sequence to the 3′ end of the one or more extension products in step (f) includes the use of a reverse transcriptase.

In some embodiments, the second primer includes RNA.

In some embodiments, the method includes removing the second target nucleic acid, or any other nucleic acid hybridized, to the one or more extension products before the complement of the second capture sequence of the one or more extension products hybridizes to the second capture domain of the second capture probe. In some embodiments, the method includes removing the first target nucleic acid, or any other nucleic acid hybridized to the ligation product, before the first capture sequence of the ligation product hybridizes to the first capture domain of the first capture probe. In some embodiments, the removing includes the use of an RNase. In some embodiments, the RNase includes RNase H. In some embodiments, the removing includes use of heat.

In some embodiments, the method includes a step of extending the 3′ end of the one or more extensions product of step (i) using the second capture probe as a template, thereby generating one or more extended capture products; and/or extending the second capture probe using the extension product of step (i) as a template, thereby generating a second extended capture probe; and/or extending the 3′ end of the ligation product using the first capture probe as a template, thereby generating an extended ligation product; and/or extending the first capture probe using the ligation product as a template, thereby generating a first extended capture probe.

In some embodiments, the extended capture product is removed from the second capture probe; and/or the extended ligation product is removed from the first capture probe.

In some embodiments, the removing includes use of heat. In some embodiments, the removing includes use of KOH.

In some embodiments, the first spatial barcode and the second spatial barcode have the same sequence. In some embodiments, the first spatial barcode and the second spatial barcode have a different sequence.

In some embodiments, the determining in step (j) includes sequencing. In some embodiments, the determining in step (k) includes sequencing. In some embodiments, the sequencing includes high-throughput sequencing.

In some embodiments, the extending in step (e) includes the use of a reverse transcriptase.

In some embodiments, the plurality of primers hybridize to different sequences in the second target nucleic acid. In some embodiments, two or more primers of the plurality of primers hybridize to different sequences in the second target nucleic acid. In some embodiments, ten or more primers of the plurality of primers hybridize to different sequences in the second target nucleic acid. In some embodiments, fifty or more primers of the plurality of primers hybridize to different sequences in the second target nucleic acid. In some embodiments, one hundred or more primers of the plurality of primers hybridize to different sequences in the second target nucleic acid.

In some embodiments, the reverse transcriptase has strand displacement activity. In some embodiments, the strand displacement activity of the reverse transcriptase displaces one or more primers of the plurality of primers from the second target nucleic acid. In some embodiments, the strand displacement activity of the reverse transcriptase displaces the one or more extension products from the second target nucleic acid.

In some embodiments, the extending in step (f) includes the use of a reverse transcriptase and a helicase. In some embodiments, the method includes use of one or more single-stranded DNA binding proteins. In some embodiments, the one or more single-stranded DNA binding proteins includes one or more of. Tth RecA, E. coli RecA, T4 gp32 and ET-SSB. In some embodiments, the helicase has strand displacement activity.

In some embodiments, the extending in step (f) includes the use of a superhelicase and a reverse transcriptase. In some embodiments, the superhelicase is selected from the group consisting of. Rep, PrcA, UvrB, RecBCD, and Tte-Uvrd.

In some embodiments, step (e) is performed after step (c). In some embodiments, step (e) is performed before step (c).

In some embodiments, the first capture sequence and the second capture sequence include the same sequence. In some embodiments, the first capture sequence and the second capture sequence include different sequences.

Also provided herein are methods for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a primer including a sequence that hybridizes to a complementary sequence in the second target nucleic acid and a functional domain; (b) hybridizing the first probe and the second probe to the first target nucleic acid; (c) generating a ligation product by ligating the first probe to the second probe; (d) hybridizing the first capture sequence of the ligation product to the first capture domain on the array, where the array includes a first plurality of capture probes, where a first capture probe of the first plurality of capture probes includes: a first spatial barcode and the first capture domain; (e) hybridizing the primer to the second target nucleic acid and extending the primer using the second target nucleic acid as a template to generate an extension product; (f) incorporating a polynucleotide sequence including at least three nucleotides to the 3′ end of the extension product; (g) hybridizing the polynucleotide sequence of the extension product to a second capture domain on an array, where the array includes a second plurality of capture probes, where a second capture probe of the second plurality of capture probes includes: a second spatial barcode and the second capture domain; (h) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and (i) determining (iii) the sequence of the second spatial barcode, or a complement thereof, and (iv) all or a portion of the sequence of the extension product corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in steps (d) and (g) include passive migration of the ligation product and/or the extension product from the biological sample to the array. In some embodiments, the hybridizing in steps (d) and (g) include active migration of the ligation product and/or the extension product from the biological sample to the array, and optionally, where the active migration includes electrophoresis.

In some embodiments, the polynucleotide sequence in step (f) includes a homopolynucleotide sequence, optionally 5′-CCC-3′. In some embodiments, the polynucleotide sequence in step (f) includes a heteropolynucleotide sequence, optionally 5′-CGC-3′.

In some embodiments, the first capture domain includes a fixed sequence. In some embodiments, the second capture domain includes a fixed sequence. In some embodiments, the first capture domain and the second capture domain have the same sequence. In some embodiments, the first capture domain and the second capture domain have a different sequence.

In some embodiments, the primer includes a random sequence. In some embodiments, the random sequence includes a random hexamer. In some embodiments, the random sequence includes a random decamer. In some embodiments, the primer includes a homopolymer sequence. In some embodiments, the homopolymer sequence includes a poly(T) sequence.

In some embodiments, the primer includes a sequence substantially complementary to a sequence in the second target nucleic acid encoding an antibody. In some embodiments, the primer includes a sequence substantially complementary to a sequence in the second target nucleic acid encoding a constant region of a B cell receptor. In some embodiments, the first primer includes a sequence substantially complementary to a sequence in the second target nucleic acid encoding a constant region of a T cell receptor.

In some embodiments, the functional domain includes a primer binding site or a sequencing specific site.

In some embodiments, the first target nucleic acid is RNA. In some embodiments, the RNA is mRNA. In some embodiments, the first target nucleic acid is DNA. In some embodiments, the second target nucleic acid is RNA. In some embodiments, the RNA is mRNA. In some embodiments, the mRNA includes a sequence encoding a B cell receptor or a fragment thereof. In some embodiments, the mRNA includes a sequence encoding a T cell receptor or a fragment thereof. In some embodiments, the mRNA includes a sequence that does not encode a B cell receptor or a fragment thereof or a T cell receptor or a fragment thereof.

In some embodiments, the extending in step (e) includes the use of a reverse transcriptase.

In some embodiments, incorporating the polynucleotide sequence to the 3′ end of the extension product in step (f) includes the use of a terminal deoxynucleotidyl transferase. In some embodiments, incorporating the polynucleotide sequence to the 3′ end of the extension product in step (f) includes the use of a reverse transcriptase.

In some embodiments, the method includes removing the first target nucleic acid, or any other nucleic acid hybridized to the ligation product, before the first capture sequence hybridizes to the first capture domain of the first capture probe; and/or removing the second target nucleic acid, or any other nucleic acid hybridized to the extension product, before the polynucleotide sequence hybridizes to the second capture domain of the second capture probe. In some embodiments, the removing includes the use of an RNase. In some embodiments, the RNase is RNase H. In some embodiments, the removing includes use of heat.

In some embodiments, the method includes a step of extending a 3′ end of the extension product of step (g) using the second capture probe as a template, thereby generating an extended capture product; and/or extending the second capture probe using the extension product of step (g) as a template, thereby generating a second extended capture probe; and/or extending the 3′ end of the ligation product using the first capture probe as a template, thereby generating an extended ligation product; and/or extending the first capture probe using the ligation product as a template, thereby generating a first extended capture probe. In some embodiments, the extended capture product is removed from the second capture probe; and/or the extended ligation product is removed from the first capture probe.

In some embodiments, the removing includes use of heat. In some embodiments, the removing includes use of KOH.

In some embodiments, the first spatial barcode and the second spatial barcode have the same sequence. In some embodiments, the first spatial barcode and the second spatial barcode have a different sequence.

In some embodiments, the determining in step (h) includes sequencing. In some embodiments, the determining in step (i) includes sequencing. In some embodiments, the sequencing includes high-throughput sequencing.

In some embodiments, step (e) is performed after step (c). In some embodiments, step (e) is performed before step (c).

In some embodiments, the first capture sequence and the second capture sequence include the same sequence. In some embodiments, the first capture sequence and the second capture sequence include different sequences.

Also provided herein are method for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a plurality of primers, where the plurality of primers include nucleic acid sequences that hybridize to complementary sequences in the second target nucleic acid and a functional domain; (b) hybridizing the first probe and the second probe to the first target nucleic acid; (c) generating a ligation product by ligating the first probe to the second probe; (d) hybridizing the ligation product to the first capture domain of the first capture probe; (e) hybridizing one or more of the plurality of primers to the second target nucleic acid and extending the one or more of the plurality of primers using the second target nucleic acid as a template to generate one or more extension products; (f) incorporating a polynucleotide sequence including at least three nucleotides to the 3′ end of the one or more extension products; (g) hybridizing the polynucleotide sequence of the one or more extension products to the second capture domain of the second capture probe; (h) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and for an extension product in the one or more extension products (i)determining (iii) the sequence of the second spatial barcode, or a complement thereof, and (iv) all or a portion of the sequence of one of the one or more extension products corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first plurality and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in steps (d) and/or (g) include passive migration of the ligation product and/or the one or more extension products from the biological sample to the array. In some embodiments, the hybridizing in steps (d) and/or (g) include active migration of the ligation product and/or the one or more extension products from the biological sample to the array, and optionally, where the active migration includes electrophoresis.

In some embodiments, incorporating the polynucleotide sequence in step (f) includes a homopolynucleotide sequence, optionally 5′-CCC-3′. In some embodiments, incorporating the polynucleotide sequence in step (f) includes a heteropolynucleotide sequence, optionally 5′-CGC-3′.

In some embodiments, the first capture domain includes a first fixed sequence. In some embodiments, the second capture domain includes a second fixed sequence.

In some embodiments, the first capture domain and the second capture domain have the same sequence. In some embodiments, the first capture domain and the second capture domain have different sequences. In some embodiments, the plurality of primers includes a random sequence. In some embodiments, the random sequence includes a random hexamer. In some embodiments, the random sequence includes a random decamer. In some embodiments, the plurality of primers includes a homopolymer sequence. In some embodiments, the homopolymer sequence includes a poly(T) sequence.

In some embodiments, the plurality of primers includes a sequence substantially complementary to a sequence in the second target nucleic acid encoding a constant region of an antibody. In some embodiments, the plurality of primers includes a sequence substantially complementary to a sequence in the second target nucleic acid encoding a constant region of a B cell receptor or a fragment thereof. In some embodiments, the plurality of primers includes a sequence substantially complementary to a sequence in the second target nucleic acid encoding a constant region of a T cell receptor or a fragment thereof.

In some embodiments, the functional domain includes a primer binding site or a sequencing specific site.

In some embodiments, the first target nucleic acid is RNA. In some embodiments, the RNA is mRNA. In some embodiments, the first target nucleic acid is DNA. In some embodiments, the second target nucleic acid is RNA. In some embodiments, the RNA is mRNA. In some embodiments, the mRNA includes a sequence encoding a T cell receptor or a fragment thereof. In some embodiments, the mRNA includes a sequence encoding a B cell receptor or a fragment thereof. In some embodiments, the mRNA includes a sequence that does not encode a B cell receptor or a fragment thereof or a T cell receptor or a fragment thereof.

In some embodiments, the extending in step (e) includes the use of a reverse transcriptase.

In some embodiments, incorporating the polynucleotide sequence to the 3′ end of the one or more extension products in step (f) includes the use of a terminal deoxynucleotidyl transferase. In some embodiments, incorporating the polynucleotide sequence to the 3′ end of the one or more extension products in step (f) includes the use of a reverse transcriptase.

In some embodiments, the method includes removing the first target nucleic acid, or any other nucleic acid hybridized to the ligation product, before the first capture sequence of the ligation product hybridizes to the first capture domain of the first capture probe. In some embodiments, the method includes removing the second target nucleic acid, or any other nucleic acid hybridized to the one or more extension products, before the polynucleotide sequence hybridizes to the second capture domain of the second capture probe. In some embodiments, the removing includes the use of an RNase. In some embodiments, the RNase is RNase H. In some embodiments, the removing includes use of heat.

In some embodiments, the method includes a step of extending a 3′ end of the one or more extension products of step (f) using the second capture probe as a template, thereby generating one or more extended capture products; and/or extending the second capture probe using the extension product of step (f) as a template, thereby generating a second extended capture probe; and/or a step of extending a 3′ end of the ligation product using the first capture probe as a template, thereby generating an extended ligation product; and/or extending the first capture probe using the ligation product as a template, thereby generating a first extended capture probe.

In some embodiments, the extended ligation product is removed from the first capture probe and/or the one or more extended capture products are removed from the second capture probes on the array. In some embodiments, the removing includes use of heat. In some embodiments, the removing includes use of KOH.

In some embodiments, the first spatial barcode and the second spatial barcode have the same sequence. In some embodiments, the first spatial barcode and the second spatial barcode have a different sequence.

In some embodiments, the determining in step (h) includes sequencing. In some embodiments, the determining in step (i) includes sequencing. In some embodiments, the sequencing includes high-throughput sequencing.

In some embodiments, the plurality of primers hybridize to different sequences in the second target nucleic acid. In some embodiments, two or more primers of the plurality of primers hybridize to different sequences in the second target nucleic acid. In some embodiments, ten or more primers of the plurality of primers hybridize to different sequences in the second target nucleic acid. In some embodiments, fifty or more primers of the plurality of primers hybridize to different sequences in the second target nucleic acid. In some embodiments, 100 or more primers of the plurality of primers hybridize to different sequences in the second target nucleic acid.

In some embodiments, the reverse transcriptase has strand displacement activity. In some embodiments, the strand displacement activity of the reverse transcriptase displaces one or more primers of the plurality of primers from the second target nucleic acid. In some embodiments, the strand displacement activity of the reverse transcriptase displaces the one or more extension products from the second target nucleic acid.

In some embodiments, the extending in step (e) includes the use of a reverse transcriptase and a helicase. In some embodiments, the method includes one or more single-stranded DNA binding proteins. In some embodiments, the one or more single-stranded DNA binding proteins includes one or more of. Tth RecA, E. coli RecA, T4 gp32 and ET-SSB.

In some embodiments, the extending in step (e) includes the use of a superhelicase and a reverse transcriptase. In some embodiments, the superhelicase is selected from the group consisting of. Rep, PrcA, UvrB, RecBCD, and Tte-Uvrd. In some embodiments, the superhelicase has strand displacement activity.

In some embodiments, the method includes generating two or more extension products from a primer of the plurality of primers. In some embodiments, the one or more extension products include different sequence lengths.

In some embodiments, the first probe and the second probe are substantially complementary to adjacent sequences of the first target nucleic acid. In some embodiments, the first probe and the second probe hybridize to sequences that are not adjacent to each other on the first target nucleic acid. In some embodiments, the first probe is extended with a DNA polymerase, thereby (i) filling in a gap between the first probe and the second probe and (ii) generating an extended first probe.

In some embodiments, contacting the first probe and the second probe with the biological sample includes contacting the biological sample with 5000 or more probe pairs, where a probe pair of the 5000 or more probe pairs includes the first probe and the second probe.

In some embodiments, generating the ligation product includes ligating the first probe to the second probe using enzymatic ligation or chemical ligation. In some embodiments, the enzymatic ligation utilizes a ligase. In some embodiments, the ligase is one or more of a T4 RNA ligase (Rnl2), a SplintR ligase (e.g., a PBCV-1 DNA Ligase, a Chlorella virus DNA ligase), a single stranded DNA ligase, or a T4 DNA ligase.

In some embodiments, the first probe includes a primer sequence.

In some embodiments, the method includes providing a blocking moiety that interacts with the first capture domain of the first capture probe. In some embodiments, the method includes releasing the blocking moiety from the first capture domain of the first capture probe prior to hybridizing the first capture sequence of the ligation product to the first capture domain.

In some embodiments, the first capture sequence includes a poly-adenylated sequence. In some embodiments, the blocking moiety includes a poly-uridine sequence, a poly-thymidine sequence, or both.

In some embodiments, the first probe and/or the second probe is a DNA probe.

In some embodiments, the blocking moiety is a DNA probe. In some embodiments, the blocking moiety from the first capture domain of the first capture probe includes contacting the biological sample with an endoribonuclease.

Also provided herein are methods for determining a location of a target nucleic acid in a biological sample, the method including: (a) contacting the biological sample with a first probe and a second probe, where: (i) the first probe includes a sequence that is substantially complementary to a sequence of the target nucleic acid and a functional domain, and (ii) the second probe includes a sequence that is substantially complementary to a sequence of the target nucleic acid and a capture sequence that is complementary to all or a portion of a capture domain; (b) hybridizing the first probe and the second probe to the target nucleic acid, where the first probe and the second probe hybridize to non-adjacent sequences; (c) generating a sequence complementary to the target nucleic acid between the first probe and second probe using the target nucleic acid as a template by gap-filling, thereby by generating an extension product; (d) ligating the extension product to the first probe or the second probe, thereby generating a ligation product; (e) hybridizing the capture sequence of the ligation product to a capture domain on an array, where the array includes a plurality of capture probes, where a capture probe of the plurality of capture probes includes a spatial barcode and the capture domain; and (f) determining (i) the sequence of the spatial barcode, or a complement thereof, and (ii) all or a portion of the sequence of the ligation product hybridized to the capture domain, or a complement thereof, and using the determined sequences of (i) and (ii) to determine the location of the target nucleic acid in the biological sample.

In some embodiments, the biological sample is disposed on the array including the plurality of capture probes. In some embodiments, the biological sample is disposed on a first substrate, and where the array including the plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in step (e) includes passive migration of the ligation product from the biological sample to the array. In some embodiments, the hybridizing in step (e) includes active migration of the ligation product from the biological sample to the array, and optionally, where the active migration includes electrophoresis.

In some embodiments, the gap-filling in step (b) includes the use of a DNA polymerase or a reverse transcriptase. In some embodiments, generating the sequence complementary to the target nucleic acid in step (c) includes extending the first probe. In some embodiments, generating the sequence complementary to the target nucleic acid in step (c) includes extending the second probe.

In some embodiments, contacting the first probe and the second probe with the biological sample includes contacting the biological sample with 5000 or more probe pairs, where a probe pair of the 5000 or more probe pairs includes the first probe and the second probe.

In some embodiments, generating the ligation product includes ligating the extension product to the first probe or the second probe using enzymatic ligation or chemical ligation. In some embodiments, the enzymatic ligation utilizes a ligase. In some embodiments, the ligase is one or more of a T4 RNA ligase (Rnl2), a SplintR ligase (e.g., a PBCV-1 DNA Ligase, a Chlorella virus DNA ligase), a single-stranded DNA ligase, or a T4 DNA ligase.

In some embodiments, the functional domain includes a primer binding sequence or a sequencing specific site.

In some embodiments, the method includes providing a blocking moiety that interacts with the capture domain of the capture probe. In some embodiments, the method includes releasing the blocking moiety from the capture domain of the capture probe.

In some embodiments, the capture sequence includes a poly-adenylated sequence. In some embodiments, the capture sequence includes a fixed sequence. In some embodiments, the fixed sequence is a homopolynucleotide sequence. In some embodiments, the fixed sequence is a heteropolynucleotide sequence.

In some embodiments, the blocking moiety includes a poly-uridine sequence, a poly-thymidine sequence, or both.

In some embodiments, the first probe and/or the second probe is a DNA probe. In some embodiments, the first probe and/or the second probe is an RNA probe. In some embodiments, the blocking moiety is a DNA probe. In some embodiments, releasing the blocking moiety from the capture domain of the capture probe includes contacting the biological sample with an endoribonuclease. In some embodiments, releasing the ligation product from the target nucleic acid includes contacting the biological sample with an endoribonuclease. In some embodiments, the endoribonuclease is RNase H.

In some embodiments, the second probe includes one or more locked nucleic acids, one or more RNA nucleotides, or both.

In some embodiments, the first probe includes a sequence complementary to a sequence in the target nucleic acid encoding a constant region of an antibody. In some embodiments, the first probe includes a sequence complementary to a sequence in the target nucleic acid encoding a constant region of a B cell receptor. In some embodiments, the first probe includes a sequence complementary to a sequence in the target nucleic acid encoding a constant region of a T cell receptor.

In some embodiments, the target nucleic acid is RNA or DNA. In some embodiments, the RNA is mRNA or the DNA is cDNA. In some embodiments, the mRNA includes a sequence encoding a B cell receptor or a fragment thereof. In some embodiments, the mRNA includes a sequence encoding a T cell receptor or a fragment thereof. In some embodiments, the mRNA includes a sequence that does not encode a B cell receptor or a fragment thereof or a T cell receptor or a fragment thereof.

In some embodiments, the second probe includes a sequence complementary to a sequence in the target nucleic acid encoding a variable region of a B cell receptor. In some embodiments, the second probe includes a sequence complementary to a sequence in the target nucleic acid encoding a variable region of a T cell receptor.

In some embodiments, the DNA polymerase or the reverse transcriptase lack strand displacement activity.

In some embodiments, the array includes one or more features.

In some embodiments, the method includes fixing the biological sample. In some embodiments, fixing the biological sample includes the use of a fixative selected from the group consisting of: ethanol, methanol, acetone, formaldehyde, paraformaldehyde-Triton, glutaraldehyde, and combinations thereof.

In some embodiments, the method includes staining the biological sample. In some embodiments, the staining includes use of eosin and/or hematoxylin. In some embodiments, the staining includes the use of a detectable label selected from the group consisting of a radioisotope, a fluorophore, a chemiluminescent compound, a bioluminescent compound, or a combination thereof.

In some embodiments, the method includes imaging the biological sample.

In some embodiments, the method includes a step of permeabilizing the biological sample. In some embodiments, the permeabilizing includes the use of an organic solvent, a detergent, an enzyme, or a combination thereof. In some embodiments, the permeabilizing includes the use of an endopeptidase, a protease, sodium dodecyl sulfate, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin or proteinase K.

In some embodiments, the capture probe, the first capture probe, and/or the second capture probe includes one or more functional domains, a unique molecular identifier, a cleavage domain, or combinations thereof.

In some embodiments, the biological sample is a tissue sample. In some embodiments, the tissue sample is a fixed tissue sample. In some embodiments, the fixed tissue sample is a formalin-fixed paraffin embedded tissue sample, a paraformaldehyde-fixed tissue sample, a methanol-fixed tissue sample, or an acetone-fixed tissue sample. In some embodiments, the tissue sample is a fresh frozen tissue sample. In some embodiments, the biological sample is a tissue section. In some embodiments, the tissue section is a fixed tissue section. In some embodiments, the fixed tissue section is a formalin-fixed paraffin embedded tissue section, a paraformaldehyde-fixed tissue section, a methanol-fixed tissue section, or an acetone-fixed tissue section. In some embodiments, the biological sample is a tissue microarray.

In some embodiments, the method includes contacting the biological sample with a third probe and a fourth probe, where: (i) the third probe includes a sequence that is substantially complementary to a sequence of a second target nucleic acid and a functional domain; (ii) the fourth probe includes a sequence that is substantially complementary to a sequence of the second target nucleic acid and a second capture sequence that is complementary to all or a portion of a second capture domain; hybridizing the third probe and the fourth probe to the second target nucleic acid; generating a second ligation product by ligating the third probe to the fourth probe; hybridizing the second capture sequence of the second ligation product to the second capture domain of a second capture probe on the array, where the second capture probe includes a second spatial barcode and the second capture domain; determining (i) the sequence of the second spatial barcode, or a complement thereof, and (ii) all or a portion of the ligation product, or a complement thereof, and using the determined sequences of (i) and (ii) to determine the location of the second target nucleic acid in the biological sample.

In some embodiments, the first capture domain and/or the second capture domain includes a fixed sequence. In some embodiments, the first capture domain and the second capture domain have the same sequence. In some embodiments, the first capture domain and the second capture domain have a different sequence.

In some embodiments, the second target nucleic acid is RNA or DNA. In some embodiments, the RNA is mRNA or the DNA is cDNA. In some embodiments, the mRNA includes a sequence encoding a B cell receptor or a fragment thereof. In some embodiments, the mRNA includes a sequence encoding a T cell receptor or a fragment thereof. In some embodiments, the mRNA includes a sequence that does not encode a B cell receptor or a fragment thereof or a T cell receptor or a fragment thereof.

In some embodiments, the method includes removing the second target nucleic acid, or any other nucleic acid hybridized to the second ligation product, before the second capture sequence hybridizes to the second capture domain of the second capture probe. In some embodiments, the removing includes the use of an RNase. In some embodiments, the RNase includes RNase H. In some embodiments, the removing includes the use of heat.

In some embodiments, the method includes a step of extending a 3′ end of the second ligation product using the second capture probe as a template, thereby generating an extended ligation product; and/or extending the second capture probe using the second ligation product as a template, thereby generating a second extended capture probe.

Also provided herein are kits including: (a) an array including a plurality of first capture probes, where a first capture probe of the plurality of capture probes includes: (i) a first spatial barcode; and (ii) a first capture domain that binds a first capture sequence; and a second plurality of capture probes, where a second capture probe of the second plurality of capture probes includes: a second spatial barcode; and a second capture domain; (b) a first primer including a sequence complementary to a target nucleic acid, and a functional domain, where the functional domain includes a primer binding sequence or a sequencing specific sequence; (c) a second primer including a second capture sequence; (d) a plurality of probes including a first probe and a second probe, where the first probe and the second probe are substantially complementary to adjacent sequences of a target nucleic acid, where the second probe includes a first capture sequence that hybridizes the first capture domain; (e) a plurality of enzymes including a ribonuclease and a ligase; and (f) instructions for performing the method of any one of claims 1-315.

In some embodiments, the second primer includes RNA. In some embodiments, the kit includes a plurality of primers, where the first primer is included in the plurality of primers.

Also provided herein are kits including: (a) an array including a plurality of first capture probes, where a first capture probe of the plurality of capture probes includes: (i) a first spatial barcode; and (ii) a first capture domain that binds a first capture sequence; and a second plurality of capture probes, where a second capture probe of the second plurality of capture probes includes: a second spatial barcode; and a second capture domain; (b) a plurality of primers, where a primer of the plurality of primers includes a sequence complementary to a target nucleic acid and a functional domain, where the functional domain is a primer binding sequence or a sequencing specific site; (c) a plurality of probes including a first probe and a second probe, where the first probe and the second probe are substantially complementary to adjacent sequences of a target nucleic acid, where the second probe includes a first capture sequence that hybridizes to the first capture domain; (e) a plurality of enzymes including a ribonuclease and a ligase; and (f) instructions for performing the method of any one of claims 1-315.

In some embodiments, the first capture probe and/or the second capture probe include one or more additional functional domains, a unique molecular identifier, a cleavage domain, and combinations thereof.

In some embodiments, the kit includes a reverse transcriptase. In some embodiments, the kit includes a polymerase.

In some embodiments, the first capture domain and/or the second capture domain includes a fixed sequence.

In some embodiments, the kit includes a helicase. In some embodiments, the kit includes one or more single-stranded DNA binding proteins selected from the group consisting of: Tth RecA, E. coli RecA, T4 gp32 and ET-SSB.

In some embodiments, the kit includes a superhelicase, where the superhelicase is selected from the group consisting of: Rep, PrcA, UvrB, RecBCD, and Tte-Uvrd.

Also provided herein are compositions including: a) a first probe and a second probe hybridized to a first target nucleic acid and ligated together, where the first probe and the second probe each include a sequence that is substantially complementary to adjacent sequences of the first nucleic acid and where one of the first probe or the second probe includes a first capture sequence; and b) a plurality of primers hybridized to a second target nucleic acid.

Also provided herein are compositions including: a) a first probe and a second probe hybridized to a first target nucleic acid and ligated together, where the first probe and the second probe each include a sequence that is substantially complementary to adjacent sequences of the first nucleic acid and where one of the first probe or the second probe includes a first capture sequence; and b) a plurality of primers hybridized to a second target nucleic acid, where the plurality of primers are extended thereby generating one or more extension products hybridized to the second target nucleic acid.

Also provided herein are compositions including: a) an array including a first plurality of capture probes and a second plurality of capture probes, where a first capture probe of the first plurality of capture probes includes a first spatial barcode and a first capture domain and where a second capture probe of the second plurality of capture probes includes a second spatial barcode and a second capture domain; b) a ligation product hybridized to the first capture domain; and c) one or more extension products hybridized to the second capture domain.

In some embodiments, the composition includes an RNase H enzyme. In some embodiments, the composition includes a ligase.

In some embodiments, the first capture probe and/or the second capture probe include a cleavage domain, one or more functional domains, a unique molecular identifier, and combinations thereof.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIGS. 2A-B are schematic diagrams showing in situ reverse transcription of a target nucleic acid with a first primer to generate an extension product and the incorporation of a non-templated polynucleotide sequence and a complement of a capture sequence into the extension product which is capable of hybridizing to a capture domain of a capture probe (FIG. 2A), and a pair of templated ligation probes and a target nucleic acid where the probes hybridize to the target nucleic acid and are ligated to generate a ligation product (FIG. 2B).

FIG. 3 is a schematic diagram showing capture and extension on an array of the extension product (e.g., cDNA product) shown in FIG. 2A and extension of the capture probe and the captured extension product (e.g., cDNA product) followed by release of the extended capture product.

FIGS. 4A-B are schematic diagrams showing reverse transcription of a target nucleic acid with a primer to generate an extension product and the incorporation of a non-templated polynucleotide sequence into the extension product which is capable of hybridizing to a capture domain of a capture probe (FIG. 4A) and a pair of templated ligation probes and a target nucleic acid where the probes hybridize to the target nucleic acid and are ligated to generate a ligation product (FIG. 4B).

FIG. 5 is a schematic diagram showing capture and extension on an array of the extension product (e.g., cDNA product) shown in FIG. 4A and extension of the capture probe and the captured extension product (e.g., cDNA product) followed by release of the extended capture product.

FIG. 6 is a schematic diagram showing reverse transcription of a target nucleic acid with a plurality of primers where the reverse transcription occurs with a reverse transcriptase with strand displacement activity or with a reverse transcriptase with a helicase or a superhelicase (top). In the embodiment shown, the primers hybridize to a region of the target nucleic acid that encodes for a constant region of an immune cell receptor and generate one or more extension products of varying lengths that include V(D)J sequences depending on where the primers hybridize to the target nucleic acid that encodes for a constant region of the immune cell receptor (bottom). This embodiment can be performed with the schematic as shown in FIG. 2A or FIG. 4A.

FIG. 7 is a schematic diagram showing capture and extension on an array of the ligation products shown in FIG. 2B and extension of the capture probe and the captured ligation product followed by release of the extended ligation product.

FIGS. 8A-B are schematic diagrams depicting exemplary sandwiching embodiments.

FIG. 8A shows an exemplary sandwiching process where a first substrate including a biological sample and a second substrate are brought into proximity with one another and a liquid reagent drop is introduced on the second substrate in proximity to the capture probes.

FIG. 8B shows a fully formed sandwich configuration creating a chamber formed from one or more spacers, the first substrate, and the second substrate including spatially barcoded capture probes.

FIGS. 9A-B are graphs showing the sequencing mapping rates for T cell receptors (FIG. 9A) and B cell receptors (FIG. 9B) from a reverse transcription reaction using a plurality of primers to the constant region and poly(A) region of target nucleic acids encoding BCRs and TCRs

FIGS. 10A-B are schematic diagrams showing a pair of templated ligation probes and a target nucleic acid where the probes hybridize to separate portions of the target nucleic acid, filling of the gap between the probes by extension of the first probe, and ligation of the extension product to the second probe (FIG. 10A). FIG. 10B is a schematic diagram showing capture and extension on an array of the ligation product shown in FIG. 10A and extension of the capture probe followed by release of the extended ligation product.

### DETAILED DESCRIPTION

The present disclosure features methods of capturing analytes on a spatial array, where the spatial array includes a plurality of capture probes and a capture probe of the plurality of capture probes includes a spatial barcode and a capture domain. Sequences more than about 1 kilobase away from the 3′ end of nucleic acid analytes are generally not captured in nucleic acid sequencing libraries, however, some analytes, such as nucleic acid analytes encoding immune cell receptors (e.g., B cell receptors, T cell receptors) contain sequences of interest (e.g., VDJ sequences) more than 1 kilobase away from the 3′ end of the analyte. Since capture on a spatial array can be biased toward capture 3′ end sequences of nucleic acid analytes (e.g., mRNA), methods are needed to capture the 5′ ends of the nucleic acid analytes, or complements thereof (e.g., a proxy of the analyte).

Analytes, such as mRNA, can be reverse transcribed by hybridizing to the target mRNA a primer that includes a sequence complementary to the target mRNA and a functional domain (e.g., a sequence domain for use in sequencing, a primer binding domain, etc.) to generate an RNA/DNA (e.g., RNA/cDNA) duplex. A polynucleotide sequence can be incorporated to the end of the cDNA. For example, a reverse transcriptase or a terminal transferase can add a polynucleotide sequence in a template-independent manner (e.g., at least three non-templated nucleotides). In some embodiments, the polynucleotide sequence is a heteropolynucleotide sequence (e.g., CGC). In some embodiments, the polynucleotide sequence is a homopolynucleotide sequence (e.g., CCC). In some embodiments, the polynucleotide sequence incorporated at the end of the cDNA is complementary to a capture domain of a capture probe. In some embodiments, the polynucleotide sequence incorporated at the end of the cDNA (e.g., a capture sequence) hybridizes to the capture domain of the capture probe. The target RNA can be removed (e.g., via digestion, denaturation, etc.) resulting in a single-stranded product that serves as a proxy of the target analyte which can be captured by a capture probe on a spatial array.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, which conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Examples of nucleic acid analytes include, but are not limited to, DNA (e.g., genomic DNA, cDNA) and RNA, including coding and non-coding RNA (e.g., mRNA, rRNA, tRNA, ncRNA). Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample can be a tissue sample. In some embodiments, a biological sample can be a tissue section. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

In some embodiments, a biological sample can be a fixed and/or stained biological sample (e.g., a fixed and/or stained tissue section). In some embodiments, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which are hereby incorporated by reference in their entirety, for a description and evaluation of PAXgene for tissue fixation. Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

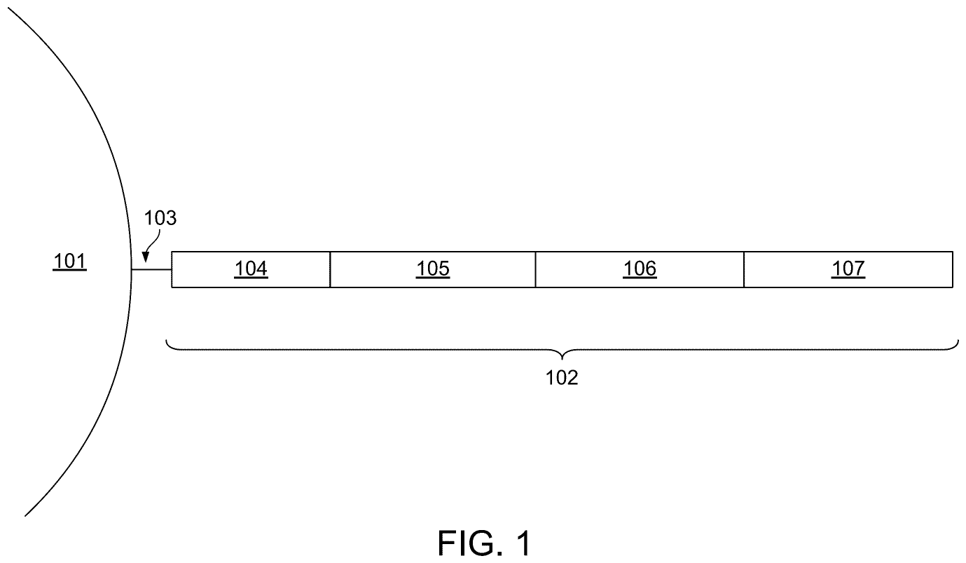

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte.

In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to an analyte capture sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, and/or an analyte capture sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature (e.g., a bead, a well, a spot on an array). In some embodiments, the UMI sequence 106 of a capture probe attached to a given feature is different from the UMI sequence of a different capture probe attached to the given feature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., an extension product, a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes incorporating (e.g., adding) to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

As used herein, “an extension product” refers to an analyte, such as RNA (e.g., mRNA), that has been reverse transcribed (e.g., with a reverse transcriptase) to generate cDNA. In some embodiments, the cDNA is hybridized to the RNA (e.g., RNA/cDNA duplex). In some embodiments, the cDNA is hybridized to the mRNA. In some embodiments, the extension product is further extended by incorporating (e.g., adding) a polynucleotide sequence (e.g., a heteropolynucleotide sequence, a homopolynucleotide sequence) to the 3′ end of the extension product (e.g., cDNA). For example, a reverse transcriptase or a terminal transferase can add at least three nucleotides (e.g., a polynucleotide sequence) to the 3′ end of the extension product in a template-independent manner. In some embodiments, the polynucleotide sequence added to the 3′ end of the extension product is complementary to a capture domain of a capture probe. In some embodiments, the polynucleotide sequence (e.g., a heteropolynucleotide sequence, a homopolynucleotide sequence) hybridizes to the capture domain of the capture probe. In some embodiments, the RNA (e.g., mRNA) is removed (e.g., by digestion) from the extension product. In such embodiments, the term extension product also refers to a single-stranded DNA (e.g., cDNA) product that includes a complement of the target nucleic acid (e.g., RNA, mRNA).

As used herein, “an extended capture product” refers to an extension product that has been captured on a spatial array and extended using the capture probe as a template. For example, an extension product, as described herein, after capture by a capture probe can be extended to include a capture sequence, or a complement thereof, that is capable of hybridizing to a capture domain of a capture probe. In some embodiments, when the extension product hybridizes to the capture domain of the capture probe, an end of the extension product (e.g., a 3′ end) can be extended to generate the extended capture product. In such examples, the extended capture product includes the domains (e.g., a UMI, a spatial barcode, one or more functional domains, or combination thereof etc.) present in the capture probe on the spatial array. In some embodiments, the extended capture product is released from the capture probe and collected for downstream applications, such as amplification and sequencing.

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe such as an extended capture product), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof), such as an extension product, can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### 5′ Capture of Target Nucleic Acids

The present disclosure features methods of capturing analytes on a spatial array, where the spatial array includes a plurality of capture probes and a capture probe of the plurality of capture probes includes a spatial barcode and a capture domain. In some embodiments, the analyte is a target nucleic acid. Since capture on a spatial array can generally be biased toward capture of the 3′ end of nucleic acid analytes, methods are needed to determine the sequence of the 5′ end of nucleic acid analytes (e.g., by capturing the 5′ end of the nucleic acid analyte), or a complement thereof (e.g., a proxy of the analyte). For example, target nucleic acid analytes (e.g., RNA) can be reverse transcribed with a primer including a sequence complementary to the target nucleic acid and a functional domain, such as a primer binding site or a sequencing specific site to generate an RNA/DNA (e.g., cDNA) duplex. An enzyme such as a reverse transcriptase or terminal transferase can add or incorporate non-templated nucleotides to the 3′ end of the cDNA. For example, a reverse transcriptase or terminal transferase enzyme can add at least 3 nucleotides (e.g., a polynucleotide sequence (e.g., a heteropolynucleotide sequence (e.g., CGC), a homopolynucleotide sequence (e.g., CCC))) to the 3′ end of the cDNA. In some embodiments, the polynucleotide sequence added to the end of the cDNA is complementary to a capture domain of a capture probe. In some embodiments, the polynucleotide sequence added to the end of the cDNA (e.g., a capture sequence) hybridizes to the capture domain of the capture probe. The target nucleic acid can be removed (e.g., digested, denatured, etc.) resulting in a single-stranded DNA product. The single-stranded DNA product can include the functional domain at its 5′ end, a copy of the target analyte (e.g., cDNA), and the polynucleotide sequence at its 3′ end that is capable of binding (e.g., hybridizing) to a capture domain of a capture probe on the array.

Alternatively, a second primer can hybridize to the polynucleotide sequence added to the 3′ end of the cDNA (e.g., extension product). The second primer can function as an extension template to further extend the extension product (e.g., a template switching oligonucleotide (TSO)). In some embodiments, the second primer includes a second capture sequence and a complement of the second capture sequence is incorporated into the extension product. In some embodiments, the second primer includes RNA. The single-stranded DNA product can include the functional domain at its 5′ end, a copy of the target analyte (e.g., cDNA), and a complement of the capture sequence that is capable of binding (e.g., hybridizing) to a capture domain of a capture probe on the array at its 3′ end.

In preferred embodiments of the methods of the disclosure, the extension reaction (e.g., reverse transcription) is performed in situ. For example, the biological sample containing the target nucleic acid is contacted with the primer or plurality of primers, such that the extension product(s) (e.g., cDNA) is generated in the biological sample.

In some embodiments, the capture domain includes a fixed sequence. As used herein, a “fixed sequence” is a non-random sequence. A “fixed sequence” can be complementary to the complement of the capture sequence that is incorporated into the extension product(s) described herein. For example, the capture domain (e.g., the fixed sequence therein) can be complementary to the non-templated polynucleotide sequence added to the extension product(s). In some embodiments, the plurality of capture probes on an array includes the same fixed sequence. The methods described herein can use different capture sequences which are incorporated (e.g., a complement thereof) into the extension product(s). For example, it is only necessary for the complement of the capture sequence (e.g., the polynucleotide sequence added to the extension products) in the extension products and the capture domain to be substantially complementary to each other, such that the capture domain including a fixed sequence can hybridize to and/or capture the extension product(s).

Target nucleic acids can include a nucleic acid molecule with a nucleic acid sequence encoding at least a portion of a V-J sequence or a V(D)J sequence of an immune cell receptor (e.g., a T cell receptor or a B cell receptor). Target nucleic acids can include a nucleic acid molecule with a nucleic acid sequence encoding an antibody. In some embodiments, the target nucleic acid is RNA. In some embodiments, the RNA is mRNA. In some embodiments, the target nucleic acids are nucleic acids encoding immune cell receptors. In some embodiments, target nucleic acids encoding immune cell receptors identify clonotype populations from a biological sample. In some embodiments, target nucleic acids include a constant region, such as a sequence encoding a constant region of an immune cell receptor (e.g., an antibody). In some embodiments, target nucleic acids include a variable region, such as a sequence encoding a variable region of an immune cell receptor (e.g., antibody), for example, a sequence encoding CDR1, CD2, and/or CDR3 of an immune cell receptor.

In some embodiments, the target nucleic acid encodes an immune cell receptor. In some embodiments, the immune cell receptor is a B cell receptor. In some embodiments, the B cell receptor includes an immunoglobulin kappa light chain. In some embodiments, the target nucleic acid includes a sequence encoding a CDR3 region of the immunoglobulin kappa light chain. In some embodiments, the target nucleic acid includes a sequence encoding one or both of CDR1 and CDR2 of the immunoglobulin kappa light chain. In some embodiments, the target nucleic acid includes a sequence encoding a full-length variable domain of the immunoglobulin kappa light chain. In some embodiments, the mRNA includes a sequence that does not encode a B cell receptor or a fragment thereof or a T cell receptor or a fragment thereof.

In some embodiments, the B cell receptor includes an immunoglobulin lambda light chain. In some embodiments, the target nucleic acid includes a sequence encoding a CDR3 of the immunoglobulin lambda light chain. In some embodiments, the target nucleic acid includes a sequence encoding one or both of CDR1 and CDR2 of the immunoglobulin lambda light chain. In some embodiments, the target nucleic acid includes a sequence encoding a full-length variable domain of the immunoglobulin lambda light chain.

In some embodiments, the B cell receptor includes an immunoglobulin heavy chain. In some embodiments, the target nucleic acid includes a sequence encoding a CDR3 of the immunoglobulin heavy chain. In some embodiments, the target nucleic acid includes a sequence encoding one or both of CDR1 and CDR2 of the immunoglobulin heavy chain. In some embodiments, the target nucleic acid includes a sequence encoding a full-length variable domain of the immunoglobulin heavy chain.

In some embodiments, the immune cell receptor is a T cell receptor. In some embodiments, the T cell receptor includes a T cell receptor alpha chain. In some embodiments, the target nucleic acid includes a sequence encoding a CDR3 of the T cell receptor alpha chain. In some embodiments, the target nucleic acid includes a sequence encoding one or both of CDR1 and CDR2 of the T cell receptor alpha chain. In some embodiments, the target nucleic acid includes a sequence encoding a full-length variable domain of the T cell receptor alpha chain.

In some embodiments, the T cell receptor includes a T cell receptor beta chain. In some embodiments, the target nucleic acid includes a sequence encoding a CDR3 of the T cell receptor beta chain. In some embodiments, the target nucleic acid includes one or both of CDR1 and CDR2 of the T cell receptor beta chain. In some embodiments, the target nucleic acid further includes a full-length variable domain of the T cell receptor beta chain.

While useful for analysis of immune cell receptor analytes such as nucleic acids encoding immune cell receptors, the disclosed methods are not limited thereto. Thus, the disclosed methods can be useful for analysis of a nucleotide sequence at and/or proximal to the 5′ end of any target nucleic acid, such as, genomic DNA, lncRNA, and the like.

In some embodiments, the methods included herein include determining the complementarity determining regions (CDRs) of a T cell receptor or a fragment thereof, and/or a B cell receptor or a fragment thereof (e.g., an antibody or fragment thereof).

Provided herein are methods for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a first primer including a sequence that hybridizes to a complementary sequence in the second target nucleic acid, and a functional domain; (b) hybridizing the first probe and the second probe to the first target nucleic acid; (c) generating a ligation product by ligating the first probe to the second probe; (d) hybridizing the first capture sequence of the ligation product to the first capture domain on an array, where the array includes a first plurality of capture probes, where a first capture probe of the first plurality of capture probes includes: a first spatial barcode and the first capture domain; (e) hybridizing the first primer to the second target nucleic acid and extending the first primer using the second target nucleic acid as a template to generate an extension product; (f) incorporating a polynucleotide sequence including at least three nucleotides to the 3′ end of the extension product; (g) hybridizing a second primer to the polynucleotide sequence of the extension product of step (f), where the second primer includes a second capture sequence; (h) extending the extension product of step (g) using the second primer as a template, thereby incorporating a complement of the second capture sequence into the extension product; (i) hybridizing the complement of the second capture sequence of the extension product of (h) to a second capture domain on the array, where the array includes a second plurality of capture probes, where a second capture probe of the second plurality of capture probes includes: a second spatial barcode and the second capture domain; (j) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and (k) determining (iii) the sequence of the second spatial barcode, or a complement thereof, and (iv) all or a portion of the sequence of the extension product corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first plurality and the second plurality of capture probes.

In some embodiments, the biological sample is disposed on the array including the first plurality and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in steps (d) and/or (i) include passive migration of the ligation product and/or the extension product from the biological sample to the array. In some embodiments, the hybridizing in steps (d) and/or (i) include active migration of the ligation product and/or the extension product from the biological sample to the array, and optionally, where the active migration includes electrophoresis.

In some embodiments, the polynucleotide sequence in step (f) includes a homopolynucleotide sequence (e.g., CCC). In some embodiments, the polynucleotide sequence in step (f) includes a heteropolynucleotide sequence (e.g., CGC).

In some embodiments, the first capture domain includes a first fixed sequence. In some embodiments, the second capture domain includes a second fixed sequence. In some embodiments, the first capture domain and the second capture domain have the same sequence. In some embodiments, the first capture domain and the second capture domain have a different sequence.

In some embodiments, the first primer includes a random sequence. In some embodiments, the random sequence includes a random hexamer. In some embodiments, the random sequence includes a random decamer. In some embodiments, the first primer includes a homopolymer sequence. In some embodiments, the homopolymer sequence includes a poly(T) sequence. In some embodiments, the first primer includes a sequence substantially complementary to a nucleic acid sequence encoding a constant region of an antibody (e.g., the second target nucleic acid). In some embodiments, the first primer includes a sequence substantially complementary to a nucleic acid sequence encoding a constant region of a B cell receptor (e.g., the second target nucleic acid). In some embodiments, the first primer includes a sequence substantially complementary to a nucleic acid sequence encoding a constant region of a T cell receptor (e.g., the second target nucleic acid).

In some embodiments, the functional domain includes a primer binding site or a sequencing specific site.

In some embodiments, the method includes removing the first target nucleic acid, or any other nucleic acid hybridized to the ligation product, before the first capture sequence of the second probe hybridizes to the first capture domain of the first capture probe. In some embodiments, the method includes removing the second target nucleic acid, or any other nucleic acid hybridized to the extension product(s) (e.g., extended cDNA product) before the sequence added to the extension product via a second primer including a sequence complementary to the added polynucleotide sequence and a second capture sequence that hybridizes to the second capture domain of the second capture probe. In some embodiments, the removing includes the use of an RNase. In some embodiments, the RNase is RNase A. In some embodiments, the RNase is RNase P. In some embodiments, the RNase is RNase T1. In some embodiments, the RNase is RNase H. In some embodiments, the removing includes heat.

In some embodiments, the extension product(s) (e.g., the single-stranded extension product(s)) hybridizes to the capture domain of the second capture probe on the substrate. In some embodiments, the 3′ end of the second capture probe is extended using the extension product as a template. In some examples, the 3′ end of the extension product (e.g., single-stranded cDNA product) is extended using the second capture probe as a template, thereby generating an extended capture product. In some embodiments, both the second capture probe, and the extension products hybridized thereto, are extended from their 3′ ends.

In some embodiments, the ligation product (e.g., the single-stranded ligation product(s)) hybridize to the capture domain of the first capture probe on the substrate. In some embodiments, the 3′ end of the first capture probe is extended using the ligation product as a template. In some embodiments, the 3′ end of the ligation product is extended using the first capture probe as a template, thereby generating an extended ligation product. In some embodiments, both the first capture probe, and the extension products hybridized thereto, are extended from their 3′ ends.

In some embodiments, step (e) is performed after step (c). In some embodiments, step (e) is performed before step (c).

Also provided herein are methods for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a primer including a sequence that hybridizes to a complementary sequence in the second target nucleic acid and a functional domain; (b) hybridizing the first probe and the second probe to the first target nucleic acid; (c) generating a ligation product by ligating the first probe to the second probe; (d) hybridizing the first capture sequence of the ligation product to the first capture domain on the array, where the array includes a first plurality of capture probes, where a first capture probe of the first plurality of capture probes includes: a first spatial barcode and the first capture domain; (e) hybridizing the primer to the second target nucleic acid and extending the primer using the second target nucleic acid as a template to generate an extension product; (f) incorporating a polynucleotide sequence including at least three nucleotides to the 3′ end of the extension product; (g) hybridizing the polynucleotide sequence of the extension product to a second capture domain on an array, where the array includes a second plurality of capture probes, where a second capture probe of the second plurality of capture probes includes: a second spatial barcode and the second capture domain; (h) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and (i) determining (iii) the sequence of the second spatial barcode, or a complement thereof, and (iv) all or a portion of the sequence of the extension product corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in steps (d) and (g) include passive migration of the ligation product and/or the extension product from the biological sample to the array. In some embodiments, the hybridizing in steps (d) and (g) include active migration of the ligation product and/or the extension product from the biological sample to the array, and optionally, where the active migration includes electrophoresis.

In some embodiments, the polynucleotide sequence in step (f) includes a homopolynucleotide sequence (e.g., CCC). In some embodiments, the polynucleotide sequence in step (f) includes a heteropolynucleotide sequence (e.g., CGC).

In some embodiments, the first capture domain includes a fixed sequence, e.g., poly(T). In some embodiments, the second capture domain includes a fixed sequence. In some embodiments, the first capture domain and the second capture domain have the same sequence, e.g., poly(T). In some embodiments, the first capture domain and the second capture domain have a different sequence. In some embodiments, the primer includes a random sequence. In some embodiments, the random sequence includes a random hexamer. In some embodiments, the random sequence includes a random decamer. In some embodiments, the primer includes a homopolymer sequence. In some embodiments, the homopolymer sequence includes a poly(T) sequence. In some embodiments, the primer includes a sequence substantially complementary to a nucleic acid sequence encoding an antibody (e.g., a second target nucleic acid). In some embodiments, first primer includes a sequence substantially complementary to a nucleic acid sequence encoding a constant region of a B cell receptor (e.g., a second target nucleic acid). In some embodiments, the first primer includes a sequence substantially complementary to a nucleic acid sequence encoding a constant region of a T cell receptor (e.g., a second target nucleic acid).

In some embodiments, the functional domain includes a primer binding site or a sequencing specific site.

In some embodiments, the extending in step (e) includes the use of a reverse transcriptase.

In some embodiments, incorporating the polynucleotide sequence to the 3′ end of the extension product in step (f) includes the use of a terminal deoxynucleotidyl transferase. In some embodiments, incorporating the polynucleotide sequence to the 3′ end of the extension product in step (f) includes the use of a reverse transcriptase.

In some embodiments, the method further includes removing the first target nucleic acid, or any other nucleic acid hybridized to the ligation product, before the first capture sequence hybridizes to the first capture domain of the first capture probe; and/or removing the second target nucleic acid, or any other nucleic acid hybridized to the extension product, before the polynucleotide sequence hybridizes to the second capture domain of the second capture probe. In some embodiments, the method includes removing the first target nucleic acid, or any other nucleic acid hybridized to the ligation product, before the first capture sequence of the second probe hybridizes to the first capture domain of the first capture probe. In some embodiments, the method includes removing the second target nucleic acid, or any other nucleic acid hybridized to the extension product(s) (e.g., extended cDNA product) before the sequence added to the extension product (e.g., a homopolynucleotide sequence, a heteropolynucleotide sequence). In some embodiments, the removing includes the use of an RNase. In some embodiments, the RNase is RNase A. In some embodiments, the RNase is RNase P. In some embodiments, the RNase is RNase T1. In some embodiments, the RNase is RNase H. In some embodiments, the removing includes use of heat.

In some embodiments, the extension product(s) (e.g., the single-stranded extension product(s)) hybridizes to the capture domain of the second capture probe on the substrate. In some embodiments, the 3′ end of the second capture probe is extended using the extension product as a template. In some examples, the 3′ end of the extension product (e.g., single-stranded cDNA product) is extended using the second capture probe as a template, thereby generating an extended capture product. In some embodiments, both the second capture probe, and the extension capture products hybridized thereto, are extended from their 3′ ends.

In some embodiments, the ligation product (e.g., the single-stranded ligation product(s)) hybridizes to the capture domain of the first capture probe on the substrate. In some embodiments, the 3′ end of the first capture probe is extended using the ligation product as a template. In some embodiments, the 3′ end of the ligation product is extended using the first capture probe as a template, thereby generating an extended ligation product. In some embodiments, both the first capture probe, and the extension products hybridized thereto, are extended from their 3′ ends.

In some embodiments, step (e) is performed after step (c). In some embodiments, step (e) is performed before step (c).

### Plurality of Primers

The methods described herein can also use a plurality of primers. For example, the plurality of primers can hybridize to a target nucleic acid at different locations in the target nucleic acid and subsequently be extended in combination with the RNA-templated ligation steps also described herein. Extending the plurality of primers generates one or more extension products that include a polynucleotide sequence that can be complementary to a capture domain of a capture probe.

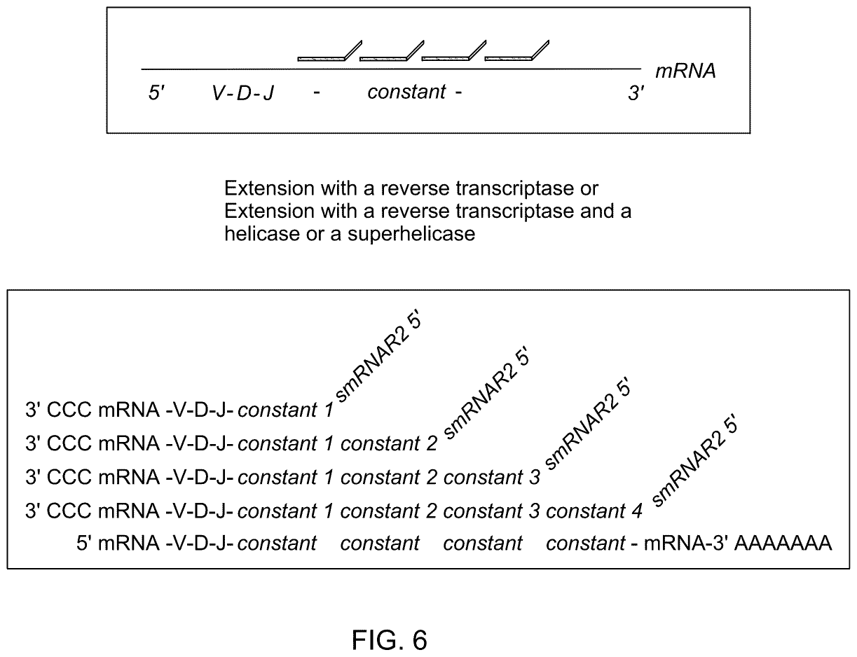

In embodiments where a plurality of primers is used, the plurality of primers can hybridize to different sequences in the target nucleic acid. For example, the plurality of primers can hybridize to adjacent sequences (e.g., “tiling”) on the target nucleic acid. Adjacent sequences can be, but are not necessarily, contiguous. In some embodiments, adjacent sequences can be within 1-10 nucleotides of each other. In some embodiments, the plurality of primers hybridize to non-adjacent sequences on the target nucleic acid. In some embodiments, two or more primers of the plurality of primers hybridize to two or more different sequences in the target nucleic acid. In some embodiments, ten or more primers of the plurality of primers hybridize to ten or more different sequences in the target nucleic acid. In some embodiments, fifty or more primers of the plurality of primers hybridize to fifty or more different sequences in the target nucleic acid. In some embodiments, one hundred or more primers of the plurality of primers hybridize to one hundred or more different sequences in the target nucleic acid. In some embodiments, a plurality of extension products can be generated by extension of the plurality of primers. The resulting extension products can have differing lengths depending on the exact location in the target nucleic acid from which they were primed. Thus, in some embodiments, extension products (e.g., cDNAs) of different lengths and/or sequences can be generated from the same target nucleic acid (e.g., mRNA). See, e.g., FIG. 6.

In some embodiments, the primer includes a sequence that is complementary to a sequence in a target nucleic acid. The sequence complementary to the target nucleic acid can be a gene specific sequence, which, for example, may allow for selective capture of a desired target nucleic acid. In some embodiments, the primer includes a homopolymer sequence and a functional domain. In some embodiments, the homopolymer sequence is a poly(T) sequence. In some embodiments, the primer includes a random sequence and a functional domain. In some embodiments, the random sequence includes 5, 6, 7, 8, 9, 10, 11, 12, 13, 14, 15, 16, 17, 18, 19, 20, or more random nucleotides. In some embodiments, the random sequence is a random hexamer. In some embodiments, the random sequence is a random decamer. In some embodiments, the functional domain is a primer binding site. In some embodiments, the functional domain is a sequencing specific site.

In some embodiments, the method includes generating two or more extension products from the primer of the plurality of primers. For example, a single primer can facilitate reverse transcription for more than a single extension reaction resulting in 2, 3, 4, 5, 6, 7, 8, 9, 10, 20, 30, 40, 50, 100, 200, 300, 400, 500, 600, 700, 800, 900, 1000, or more extension products produced from a single primer. In some embodiments, the two or more extension products generated from the same primer include the same sequence and/or length.

In some embodiments, the plurality of primers includes multiple unique primers, where each unique primer includes a unique sequence. The plurality of primers may include multiple copies (e.g., 2, 10, 20, 50, 100, 1000, 10000 or more) of each unique primer. In some embodiments, each unique primer in the plurality of primers hybridizes to a distinct sequence within the target nucleic acid. Thus, in some embodiments, the one or more extension products include different sequences and/or lengths. For example, when a plurality of primers is hybridized to a target nucleic acid and extended, the one or more extension products can have varying sequences and/or lengths.

In embodiments where the target nucleic acid includes a sequence that encodes an immune cell receptor, the plurality of primers preferably hybridize to a region of the target nucleic acid that encodes a constant region of the immune cell receptor.

In some embodiments, the extension product(s) that hybridize to the capture domains of the plurality of capture probes can migrate (e.g., diffuse) towards the capture probes through passive migration such as gravity. In some embodiments, the extension product(s) that hybridize to the capture domains of the plurality of capture probes can migrate toward the capture probes through active migration. In some embodiments, the active migration includes electrophoresis.

In some embodiments, the array includes one or more features (e.g., any of the features described herein). In some embodiments, the one or more features includes a bead.

As described herein, a capture probe refers to any molecule capable of capturing (directly or indirectly) and/or labelling an analyte (e.g., an analyte of interest, such as a nucleic acid) in a biological sample. In some embodiments, the capture probe is a nucleic acid. In some embodiments, the capture probe is DNA. In some embodiments, the capture probe includes a barcode (e.g., a spatial barcode) and/or a unique molecular identifier (UMI)) and a capture domain. In some embodiments, a capture probe can include a cleavage domain and/or one or more functional domains (e.g., a primer-binding site or a sequencing specific site, such as for next-generation sequencing (NGS)).

Thus provided herein are methods for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a plurality of primers, where the plurality of primers includes nucleic acid sequences that hybridize to complementary sequences in the second target nucleic acid and a functional domain; (b) hybridizing the first probe and the second probe to the first target nucleic acid; (c) generating a ligation product by ligating the first probe to the second probe; (d) hybridizing the first capture sequence of the ligation product to a first capture domain on an array, where the array includes a first plurality of capture probes, where a first capture probe of the plurality of first capture probes includes: a first spatial barcode and the first capture domain; (e) hybridizing one or more of the plurality of primers to the second target nucleic acid and extending the one or more of the plurality of primers using the second target nucleic acid as a template to generate one or more extension products; (f) incorporating a polynucleotide sequence including at least three nucleotides to the 3′ end of the one or more extension products; (g) hybridizing a second primer to the polynucleotide sequence of the one or more extension products of step (f), where the second primer includes a second capture sequence; (h) extending the one or more extension products of step (g) using the second primer as a template, thereby incorporating a complement of the second capture sequence into the one or more extension products; (i) hybridizing the complement of the second capture sequence of the one or more extension products of (h) to a second capture domain on the array, where the array includes a second plurality of capture probes, where a second capture probe of the second plurality of capture probes includes: a second spatial barcode and the second capture domain; (j) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and for an extension product in the one or more extension products (k) determining (iii) the sequence of the second spatial barcode, or a complement thereof, and (iv) all or a portion of the sequence of one of the one or more extension products corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first plurality and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in steps (d) and/or (i) include passive migration of the ligation product and/or the one or more extension products from the biological sample to the array. In some embodiments, the hybridizing in steps (d) and/or (i) include active migration of the ligation product and/or the one or more extension products from the biological sample to the array, and optionally, where the active migration includes electrophoresis.

In some embodiments, incorporating the polynucleotide sequence in step (f) includes a homopolynucleotide sequence (e.g., CCC). In some embodiments, incorporating the polynucleotide sequence in step (f) includes a heteropolynucleotide sequence (e.g., CGC).

In some embodiments, the first capture domain includes a fixed sequence. In some embodiments, the second capture domain includes a fixed sequence. In some embodiments, the first capture domain and the second capture domain have the same sequence. In some embodiments, the first capture domain and the second capture domain have a different sequence. In some embodiments, the primer includes a random sequence. In some embodiments, the random sequence includes a random hexamer. In some embodiments, the random sequence includes a random decamer. In some embodiments, the primer includes a homopolymer sequence. In some embodiments, the homopolymer sequence includes a poly(T) sequence. In some embodiments, the primer includes a sequence substantially complementary to a nucleic acid sequence encoding an antibody (e.g., a second target nucleic acid). In some embodiments, first primer includes a sequence substantially complementary to a nucleic acid sequence encoding a constant region of a B cell receptor (e.g., a second target nucleic acid). In some embodiments, the first primer includes a sequence substantially complementary to a nucleic acid sequence encoding a constant region of a T cell receptor (e.g., a second target nucleic acid).

In some embodiments, the functional domain includes a primer binding site or a sequencing specific site.

In some embodiments, incorporating the polynucleotide sequence to the 3′ end of the extension product in step (f) includes the use of a terminal deoxynucleotidyl transferase. In some embodiments, incorporating the polynucleotide sequence to the 3′ end of the extension product in step (f) includes the use of a reverse transcriptase.

In some embodiments, the second primer includes RNA.

In some embodiments, the method includes removing the second target nucleic acid, or any other nucleic acid hybridized, to the one or more extension products before the second capture sequence of the second primer hybridizes to the second capture domain of the second capture probe.

In some embodiments, the method includes removing the first target nucleic acid, or any other nucleic acid hybridized to the ligation product, before the first capture sequence of the second probe hybridizes to the first capture domain of the first capture probe. In some embodiments, the removing includes the use of an RNase. In some embodiments, the RNase is RNase A. In some embodiments, the RNase is RNase P. In some embodiments, the RNase is RNase T1. In some embodiments, the RNase is RNase H. In some embodiments, the removing includes heat.

In some embodiments, the method includes a step of extending the 3′ end of the one or more extensions product of step (g) using the second capture probe as a template, thereby generating an extended capture product; and/or extending the 3′ end of the ligation product using the first capture probe as a template, thereby generating an extended ligation product.

In some embodiments, the extended capture product is removed from the second capture probe; and/or the extended ligation product is removed from the first capture probe.

In some embodiments, the removing includes the use of an RNase. In some embodiments, the RNase is RNase A. In some embodiments, the RNase is RNase P. In some embodiments, the RNase is RNase T1. In some embodiments, the RNase is RNase H. In some embodiments, the removing includes heat.

In some embodiments, the extension product(s) (e.g., the single-stranded extension product(s)) hybridizes to the capture domain of the second capture probe on the substrate. In some embodiments, the 3′ end of the second capture probe is extended using the extension product as a template. In some examples, the 3′ end of the extension product (e.g., single-stranded cDNA product) is extended using the second capture probe as a template, thereby generating an extended capture product. In some embodiments, both the second capture probe, and the extension products hybridized thereto, are extended from their 3′ ends.

In some embodiments, the ligation product (e.g., the single-stranded ligation product(s)) hybridizes to the capture domain of the first capture probe on the substrate. In some embodiments, the 3′ end of the first capture probe is extended using the ligation product as a template. In some embodiments, the 3′ end of the ligation product is extended using the first capture probe as a template, thereby generating an extended ligation product. In some embodiments, both the first capture probe, and the extension products hybridized thereto, are extended from their 3′ ends.

In some embodiments, step (e) is performed after step (c). In some embodiments, step (e) is performed before step (c).

Also provided herein are method for determining a location of a first target nucleic acid and a second target nucleic acid in a biological sample, the method including: (a) contacting the biological sample with: (i) a first probe and a second probe, where the first probe and the second probe each include sequences that are substantially complementary to sequences of the first target nucleic acid, and where the second probe includes a first capture sequence that is complementary to all or a portion of a first capture domain; and (ii) a plurality of primers, where the plurality of primers include nucleic acid sequences that hybridize to complementary sequences in the second target nucleic acid and a functional domain; (b) hybridizing the first probe and the second probe to the first target nucleic acid; (c) generating a ligation product by ligating the first probe to the second probe; (d) hybridizing the ligation product to the first capture domain of the first capture probe; (e) hybridizing one or more of the plurality of primers to the second target nucleic acid and extending the one or more of the plurality of primers using the second target nucleic acid as a template to generate one or more extension products; (f) incorporating a polynucleotide sequence including at least three nucleotides to the 3′ end of the one or more extension products; (g) hybridizing the polynucleotide sequence of the one or more extension products to the second capture domain of the second capture probe; (h) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and for an extension product in the one or more extension products (i) determining (iii) the sequence of the second spatial barcode, or a complement thereof, and (iv) all or a portion of the sequence of one of the one or more extension products corresponding to a sequence of the second target nucleic acid, or a complement thereof, and using the determined sequences of (iii) and (iv) to determine the location of the second target nucleic acid in the biological sample.

In some embodiments, the biological sample is disposed on the array including the first plurality and the second plurality of capture probes. In some embodiments, the biological sample is disposed on a first substrate, and where the array including the first and second plurality of capture probes is disposed on a second substrate. In some embodiments, the method includes aligning the first substrate with the second substrate, such that at least a portion of the biological sample is aligned with at least a portion of the second substrate.

In some embodiments, the hybridizing in steps (d) and/or (g) include passive migration of the ligation product and/or the one or more extension products from the biological sample to the array. In some embodiments, the hybridizing in steps (d) and/or (g) include active migration of the ligation product and/or the one or more extension products from the biological sample to the array, and optionally, where the active migration includes electrophoresis.

In some embodiments, incorporating the polynucleotide sequence in step (f) includes a homopolynucleotide sequence (e.g., CCC). In some embodiments, incorporating the polynucleotide sequence in step (f) includes a heteropolynucleotide sequence (e.g., CGC).

In some embodiments, the first capture domain includes a first fixed sequence. In some embodiments, the second capture domain includes a second fixed sequence. In some embodiments, the first capture domain and the second capture domain have the same sequence. In some embodiments, the first capture domain and the second capture domain have different sequences.

In some embodiments, the plurality of primers includes a random sequence. In some embodiments, the random sequence includes a random hexamer. In some embodiments, the random sequence includes a random decamer. In some embodiments, the plurality of primers includes a homopolymer sequence. In some embodiments, the homopolymer sequence includes a poly(T) sequence. In some embodiments, the plurality of primers includes a sequence substantially complementary to a nucleic acid sequence encoding an antibody (e.g., a second target nucleic acid). In some embodiments, the plurality of primers includes a sequence substantially complementary to a nucleic acid sequence encoding a constant region of a B cell receptor (e.g., a second target nucleic acid). In some embodiments, the plurality of primers includes a sequence substantially complementary to a nucleic acid sequence encoding a constant region of a T cell receptor (e.g., a second target nucleic acid).

In some embodiments, the functional domain includes a primer binding site or a sequencing specific site.

In some embodiments, the extending in step (e) includes the use of a reverse transcriptase. In some embodiments, incorporating the polynucleotide sequence to the 3′ end of the extension product in step (f) includes the use of a terminal deoxynucleotidyl transferase. In some embodiments, incorporating the polynucleotide sequence to the 3′ end of the extension product in step (f) includes the use of a reverse transcriptase.

In some embodiments, the method includes removing the first target nucleic acid, or any other nucleic acid hybridized to the ligation product, before the first capture sequence of the second probe hybridizes to the first capture domain of the first capture probe.

In some embodiments, the method includes removing the second target nucleic acid, or any other nucleic acid hybridized to the one or more extension products, before the polynucleotide sequence hybridizes to the second capture domain of the second capture probe.

In some embodiments, the removing includes the use of an RNase. In some embodiments, the RNase is RNase A. In some embodiments, the RNase is RNase P. In some embodiments, the RNase is RNase T1. In some embodiments, the RNase is RNase H. In some embodiments, the removing includes heat.

In some embodiments, the extending in step (e) includes the use of a reverse transcriptase. In some embodiments, the reverse transcriptase has strand displacement activity. In some embodiments, the strand displacement activity of the reverse transcriptase displaces one or more primers of the plurality of primers from the target nucleic acid. In some embodiments, the strand displacement activity of the reverse transcriptase displaces the one or more extension products from the target nucleic acid.

In some embodiments, the extending in step (e) includes the use of a reverse transcriptase and a helicase.

In some embodiments, step (e) is performed after step (c). In some embodiments, step (e) is performed before step (c).

In some embodiments, the extending in step (e) includes the use of a reverse transcriptase (e.g., any suitable reverse transcriptase known in the art). In some embodiments, incorporating (e.g., adding) the polynucleotide sequence to the 3′ end of the extension product in step (f) includes the use of the reverse transcriptase. In some embodiments, a second a primer hybridizes to the added polynucleotide sequence and the extension product can be further extended using the second primer as an extension template where the second primer includes a second capture sequence. In such embodiments, the extension product is further extended, thereby incorporating a complement of the second capture sequence into one or more extension products. In some embodiments, incorporating the polynucleotide sequence to the 3′ end of the extension product in step (f) includes the use of a terminal transferase. In some embodiments, the terminal transferase is terminal deoxynucleotidyl transferase. In some embodiments, the reverse transcriptase or the terminal transferase adds at least three nucleotides to the extension product. In some embodiments, the reverse transcriptase or the terminal transferase adds 4, 5, 6, 7, 8, 9, 10, or more nucleotides (e.g., a polynucleotide sequence) to the 3′ end of the extension product(s) in step (c). In some embodiments, the polynucleotide sequence added to the 3′ end of the extension product(s) is 5′-CCC-3′. In some embodiments, the polynucleotide sequence added to the 3′ end of the extension product(s) is 5′-CGC-3′.

In some embodiments, step (e) is performed after step (c). In some embodiments, step (e) is performed before step (c).

As defined herein, “helicases” are enzymes that catalyze the reaction of separating or unwinding the helical structure of nucleic acid complexes, e.g., double-stranded DNA, double-stranded RNA, or DNA:RNA complexes, into single stranded components. Helicases generally are known to use a nucleoside triphosphate (NTP) (e.g., ATP) hydrolysis as a source of energy. In some embodiments, the method includes one or more single-stranded DNA binding proteins. In some embodiments, the one or more single-stranded DNA binding proteins includes one or more of: Tth RecA, E. coli RecA, T4 gp32 and ET-SSB. As defined herein, “single-stranded DNA binding proteins” or “SSBs” are proteins that bind to single-stranded DNA. SSBs, or functional equivalents, are found in a variety of organisms, including eukaryotes and bacteria. Single-stranded DNA is produced, for example, during aspects of DNA metabolism, DNA replication, DNA recombination, and DNA repair. As well as stabilizing single-stranded DNA, SSB proteins bind to and modulate the function of numerous proteins involved in the aforementioned processes. In addition, SSB proteins can destabilize ends of double-stranded nucleic acid (e.g., dsDNA). In some embodiments, the helicase has strand displacement activity. For example, the helicases and single-stranded DNA binding proteins described herein can unwind DNA:RNA complexes that allow a reverse transcriptase to reverse transcribe the target nucleic acid. In some embodiments, a helicase can unwind a first target nucleic acid: extension product complex (e.g., mRNA:cDNA) that is downstream of a second target nucleic acid: extension product complex, such that the target nucleic acid from the first complex (with or without the help of the SSBs) becomes available as template for further extension of the extension product in the second complex.

In some embodiments, helicases, including superhelicases, are also used in conjunction with SSB proteins during nucleic acid amplification. In some embodiments, the extending in step (e) includes the use of a superhelicase and a reverse transcriptase. In some embodiments, the superhelicase is selected from the group consisting of: Rep, PrcA, UvrB, RecBCD, and Tte-Uvrd. As defined herein a “superhelicase” is a mutant and/or a derivative of a helicase. Superhelicases can have increased processivity compared to helicases due to one or more derivations that can include mutated gene or substituted polypeptide sequences and/or cross-linked protein domains. Additionally, superhelicases can unwind double-stranded nucleic acid complexes without single-stranded DNA binding protein(s). In some embodiments, the helicase is a superhelicase. In some embodiments, superhelicases have increased processivity relative to helicases. In some embodiments, the superhelicase has strand displacement activity. For example, superhelicases can unwind double-stranded nucleic acid complexes longer than 150 base pairs.

In some embodiments, the extending includes the use of a polymerase. Any suitable polymerase can be used (e.g., Kapa HiFi). In some examples, the 3′ end of the first and/or second capture probe is extended using the ligation product or extension product as a template and the 3′ end of the ligation product or extension product is simultaneously extended using the first capture probe or second capture probe as a template (e.g., generating an extended ligation product and an extended capture product, respectively). In some examples, the extended ligation product and/or the extended capture product is released from the first or second capture probe. In some examples, the extended ligation product and/or the extended capture product is released via heat. In some examples, the extended ligation product and/or the extended capture product is denatured from the first or second capture probe. In some examples, the extended ligation product and/or the extended capture product is denatured from the first or second capture probe with KOH.

In some embodiments, the first spatial barcode and the second spatial barcode have the same sequence. In some embodiments, the first spatial barcode and the second spatial barcode have a different sequence.

In some embodiments, the released, extended ligation products and/or the extended captured products can be prepared for downstream applications, such as generation of a sequencing library and next-generation sequencing. Generating sequencing libraries are known in the art. For example, the extended ligation products and the extended captured products can be purified and collected for downstream amplification steps. The extended amplification products (e.g., from either or both of the extended ligation products and/or the extended capture products) can be amplified using PCR, where primer binding sites flank the spatial barcode and target nucleic acid (e.g., the first target nucleic acid, the second target nucleic acid), or a complement thereof, generating a library associated with a particular spatial barcode. In some embodiments, the library preparation can be quantitated and/or quality controlled to verify the success of the library preparation steps. The library amplicons are sequenced and analyzed to decode spatial information and the target nucleic acid sequence.

In some embodiments, the method includes generating a sequencing library. In some embodiments, the determining includes sequencing. Methods and systems for sequencing are known in the art and are described herein. In some embodiments, the sequencing is high-throughput sequencing. In some embodiments, the sequencing is long read sequencing.

Alternatively, or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flow cell (e.g., on Illumina sequencing instruments). Additionally, i7 and i5 can index sequences be added as sample indexes if multiple libraries are to be pooled and sequenced together. Further, Read 1 and Read 2 sequences can be added to the library preparation for sequencing purposes. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

### Templated-Ligation

Targeted nucleic acid (e.g., DNA, RNA) capture can be an alternative to traditional capture methods (e.g., poly(A) capture) in order to interrogate spatial gene expression in a biological sample. However, as described herein targeted nucleic acid capture (e.g., template-ligation capture) can be used in conjunction with traditional capture methods. Generally, targeted nucleic capture is less affected by degradation and allows for sensitive measurement of specific genes of interest that otherwise might be missed with a whole transcriptomic approach. Targeted nucleic acid capture can be used to capture a defined set of nucleic acid molecules of interest, or it can be used at a whole transcriptome level, or anything in between. When combined with the spatial methods disclosed herein, the location and abundance of the nucleic acid targets can be determined.

In some embodiments, the methods described herein include a first probe. As used herein, a “first probe” and “second probe” can refer to a probe that hybridizes to all or a portion of a target nucleic acid and can be ligated to one or more additional probes (e.g., a second probe or a spanning probe). In some embodiments, “first probe” can be used interchangeably with “first probe oligonucleotide.” In some embodiments, “second probe” can be used interchangeably with “second probe oligonucleotide.” In some embodiments, the first probe and/or the second probe is a DNA probe.

In some embodiments, the first probe and/or the second probe includes ribonucleotides, deoxyribonucleotides, and/or synthetic nucleotides that are capable of participating in Watson-Crick type or analogous base pair interactions. In some embodiments, the first probe and/or the second probe includes deoxyribonucleotides. In some embodiments, the first probe and/or the second probe includes deoxyribonucleotides and ribonucleotides. In some embodiments, the first probe and/or the second probe includes a deoxyribonucleic acid that hybridizes to an analyte and includes a portion of the oligonucleotide that is not a deoxyribonucleic acid. For example, in some embodiments, the portion of the first oligonucleotide and/or the second oligonucleotide that is not a deoxyribonucleic acid is a ribonucleic acid or any other non-deoxyribonucleic acid nucleic acid as described herein. In some embodiments where the first probe and/or the second probe includes deoxyribonucleotides, hybridization of the first probe and/or the second probe to the nucleic acid (e.g., RNA, mRNA, etc.) molecule results in a DNA:RNA hybrid. In some embodiments, the first probe and/or the second probe includes only deoxyribonucleotides and upon hybridization of the first probe and/or the second probe to the mRNA molecule results in a DNA:RNA hybrid.

In some embodiments, the method includes a first probe and/or a second probe that includes one or more sequences that are substantially complementary to one or more sequences of an analyte. In some embodiments, a first probe includes a sequence that is substantially complementary to a first target sequence in the first target nucleic acid. In some embodiments, the sequence of the first probe that is substantially complementary to the first target sequence in the first target nucleic acid is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the first target nucleic acid. In some embodiments, the sequence of the second probe that is substantially complementary to the second target sequence in the first target nucleic acid is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence in the first target nucleic acid.

In some embodiments, a first probe and/or a second probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a sequence of the first probe and/or the second probe that is substantially complementary to a sequence in the target nucleic acid includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some embodiments, a first probe includes a functional sequence. In some embodiments, a functional sequence includes a primer sequence.

In some embodiments, a second probe includes a “first capture sequence.” As used herein, a “first capture sequence” is a sequence, domain, or moiety that can bind specifically to a capture domain of a capture probe (e.g., the first capture domain of a first capture probe). In some embodiments, a second probe includes a sequence from 5′ to 3′: a sequence that is substantially complementary to a sequence in the analyte and a first capture sequence.

In some embodiments, a first capture sequence includes a poly(A) sequence. In some embodiments, the first capture sequence includes a poly-uridine sequence, a poly-thymidine sequence, or both. In some embodiments, the first capture sequence includes a random sequence (e.g., a random hexamer or octamer). In some embodiments, the first capture sequence is complementary to a capture domain in a capture probe that detects a particular target nucleic acid.

In some embodiments, a blocking moiety that interacts with the first capture domain is provided. In some embodiments, a blocking moiety includes a sequence that is complementary or substantially complementary to a first capture domain. In some embodiments, a blocking moiety prevents the first capture sequence from binding the capture probe when present. In some embodiments, a blocking moiety is removed prior to binding the first capture sequence (e.g., present in a ligated probe) to a first capture probe. In some embodiments, a blocking moiety includes a poly-uridine sequence, a poly-thymidine sequence, or both. In some embodiments, the first capture sequence includes ribonucleotides, deoxyribonucleotides, and/or synthetic nucleotides that are capable of participating in Watson-Crick type or analogous base pair interactions. In some embodiments, the capture domain sequence includes at least 10, 11, 12, 13, 14, 15, 16, 17, 18, 19, or 20 nucleotides. In some embodiments, the capture domain sequence includes at least 25, 30, or 35 nucleotides.

In some embodiments, the method includes releasing the blocking moiety from the first capture domain of the first capture probe prior to hybridizing the first capture sequence of the ligation product to the first capture domain. In some embodiments, releasing the blocking moiety from the first capture domain of the first capture probe includes contacting the biological sample with an endoribonuclease. In some embodiments, releasing the ligation product from the first target nucleic acid includes contacting the biological sample with an endoribonuclease. In some embodiments, the endoribonuclease is an RNase H enzyme.

In some embodiments, after hybridization of the probe oligonucleotides (e.g., a first probe, a second probe) to the target nucleic acid the probe (e.g., a first probe, a second probe) can be ligated together, creating a single ligation product that includes one or more sequences that are complementary to the analyte. Ligation can be performed enzymatically or chemically, as described herein.

In some instances, the ligation is an enzymatic ligation reaction, using a ligase (e.g., T4 RNA ligase (Rnl2), a SplintR ligase (also known as PBCV-1 DNA ligase or Chlorella virus DNA ligase), a single stranded DNA ligase, or a T4 DNA ligase). See, e.g., Zhang et al.; RNA Biol. 2017; 14(1): 36-44, which is incorporated by reference in its entirety, for a description of KOD ligase. Following the enzymatic ligation reaction, the probes can be considered ligated.

In some embodiments, the first probe and the second probe hybridize to sequences that are not adjacent to each other on the analyte (e.g., nucleic acid). In some embodiments, the complementary sequences to which the first probe and the second probe hybridize are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, or about 150 nucleotides away from each other. Gaps between the probes may first be filled prior to coupling (e.g., ligation), using, for example, dNTPs in combination with a polymerase such as polymerase mu, DNA polymerase, RNA polymerase, reverse transcriptase, VENT polymerase, Taq polymerase, and/or any combinations, derivatives, and variants (e.g., engineered mutants) thereof. In some embodiments, when the first and second probes are separated from each other by one or more nucleotides, deoxyribonucleotides are used to extend and couple (e.g., ligate) the first and second probe oligonucleotides. In some embodiments, the first probe and the second probe hybridize to sequences that are at least 1, 2, 3, 4, 5, 6, 7, 8, 9, 10, or more nucleotides away from one another.

In some embodiments, the first probe is extended with a DNA polymerase, thereby (i) filling in a gap between the first probe and the second probe and (ii) generating an extended first probe. In some embodiments, the second probe is extended with a DNA polymerase, thereby (i) filling in a gap between the first probe and the second probe and (ii) generating an extended second probe.

In some embodiments, about 1000, 5000, 10000, 15000, 20,000 or more probe pairs (e.g., a probe pair including a first probe and a second probe) are used in the methods described herein. For example, contacting the first probe and the second probe with the biological sample includes contacting the biological sample with 5000 or more probe pairs, where a probe pair of the 5000 or more probe pairs includes the first probe and the second probe.

### Gap-Filling Method

Also provided herein are methods for determining a location of a target nucleic acid in a biological sample, the method including: (a) contacting the biological sample with a first probe and a second probe, where: (i) the first probe includes a sequence that is substantially complementary to a sequence of the target nucleic acid and a functional domain, and (ii) the second probe includes a sequence that is substantially complementary to a sequence of the target nucleic acid and a capture sequence that is complementary to all or a portion of a capture domain; (b) hybridizing the first probe and the second probe to the target nucleic acid, where the first probe and the second probe hybridize to non-adjacent sequences; (c) generating a sequence complementary to the target nucleic acid between the first probe and second probe using the target nucleic acid as a template by gap-filling, thereby by generating an extension product; (d) ligating the extension product to the first probe or the second probe, thereby generating a ligation product; (e) hybridizing the capture sequence of the ligation product to a capture domain on an array, where the array includes a plurality of capture probes, where a capture probe of the plurality of capture probes includes a spatial barcode and the capture domain; and (f) determining (i) the sequence of the spatial barcode, or a complement thereof, and (ii) all or a portion of the sequence of the ligation product hybridized to the capture domain, or a complement thereof, and using the determined sequences of (i) and (ii) to determine the location of the target nucleic acid in the biological sample.

In some embodiments, the biological sample is disposed on the array including the plurality of capture probes. In some embodiments, the biological sample is disposed on a first substrate, and where the array including the plurality of capture probes is disposed on a second substrate. In some embodiments, the method includes aligning the first substrate with the second substrate, such that at least a portion of the biological sample is aligned with at least a portion of the array on the second substrate.

In some embodiments, the hybridizing in steps (e) includes passive migration of the ligation product from the biological sample to the array. In some embodiments, the hybridizing in steps (e) includes active migration of the ligation product from the biological sample to the array, and optionally, where the active migration includes electrophoresis.

In some embodiments, the gap-filling in step (b) includes the use of a DNA polymerase or a reverse transcriptase. In some embodiments, generating the sequence complementary to the target nucleic acid in step (c) includes extending the first probe. In some embodiments, generating the sequence complementary to the target nucleic acid in step (c) includes extending the second probe.

In some embodiments, contacting the first probe and the second probe with the biological sample includes contacting the biological sample with 5000 or more probe pairs, where a probe pair of the 5000 or more probe pairs includes the first probe and the second probe.

In some embodiments, generating the ligation product includes ligating the extension product to the second probe using enzymatic ligation or chemical ligation. In some embodiments, the enzymatic ligation utilizes a ligase. In some embodiments, the ligase is one or more of a T4 RNA ligase (Rnl2), a SplintR ligase, a single stranded DNA ligase, or a T4 DNA ligase.

In some embodiments, the functional domain includes a primer binding sequence or a sequencing specific site.

In some embodiments, the method includes providing a blocking moiety that interacts with the capture domain of the capture probe. In some embodiments, the blocking moiety is a DNA probe. In some embodiments, the method includes releasing the blocking moiety from the capture domain of the first capture probe. In some embodiments, the blocking moiety includes a poly-uridine sequence, a poly-thymidine sequence, or both. In some embodiments, releasing the blocking moiety from the capture domain of the capture probe includes contacting the biological sample with an endoribonuclease. In some embodiments, releasing the ligation product from the target nucleic acid includes contacting the biological sample with an endoribonuclease. In some embodiments, the endoribonuclease is RNase H.

In some embodiments, the capture sequence includes a poly-adenylated sequence. In some embodiments, the capture sequence includes a fixed sequence. In some embodiments, the fixed sequence is a homopolynucleotide sequence. In some embodiments, the fixed sequence is a heteropolynucleotide sequence.

In some embodiments, the first probe and/or the second probe is a DNA probe. In some embodiments, the first probe and/or the second probe is an RNA probe. In some embodiments, the second probe includes one or more locked nucleic acids, one or more RNA nucleotides, or both.

In some embodiments, the first probe includes a sequence substantially complementary to a nucleic acid sequence encoding a constant region of an antibody. In some embodiments, the first probe includes a sequence substantially complementary to a nucleic acid sequence encoding a constant region of a B cell receptor. In some embodiments, the first probe includes a sequence substantially complementary to a nucleic acid sequence encoding a constant region of a T cell receptor. In some embodiments, the second probe includes a sequence substantially complementary to a nucleic acid sequence encoding a variable region of a B cell receptor. In some embodiments, the second probe includes a sequence substantially complementary to a nucleic acid sequence encoding a variable region of a T cell receptor.

In some embodiments, the DNA polymerase or the reverse transcriptase lack strand displacement activity.

In some embodiments, the target nucleic acid is RNA. In some embodiments, the RNA is mRNA. In some embodiments, the mRNA includes a sequence encoding a B cell receptor. In some embodiments, the mRNA includes a sequence encoding a T cell receptor. In some embodiments, the mRNA includes a sequence that does not encode a B cell receptor or a fragment thereof or a T cell receptor or a fragment thereof.

In some embodiments, the method includes contacting the biological sample with a third probe and a fourth probe, where: (i) the third probe includes a sequence that is substantially complementary to a sequence of a second target nucleic acid and a functional domain; (ii) the fourth probe includes a sequence that is substantially complementary to a sequence of the second target nucleic acid and a second capture sequence that is complementary to all or a portion of a second capture domain; hybridizing the third probe and the fourth probe to the second target nucleic acid; generating a second ligation product by ligating the third probe to the fourth probe; hybridizing the second capture sequence of the second ligation product to the second capture domain of a second capture probe on the array, where the second capture probe includes a second spatial barcode and the second capture domain; determining (i) the sequence of the second spatial barcode, or a complement thereof, and (ii) all or a portion of the ligation product, or a complement thereof, and using the determined sequences of (i) and (ii) to determine the location of the second target nucleic acid in the biological sample.

In some embodiments, the first capture domain and/or the second capture domain includes a fixed sequence. In some embodiments, the first capture domain and the second capture domain have the same sequence. In some embodiments, the first capture domain and the second capture domain have a different sequence.

In some embodiments, the second target nucleic acid is RNA or DNA. In some embodiments, the RNA is mRNA or the DNA is cDNA. In some embodiments, the mRNA includes a sequence encoding a B cell receptor or a fragment thereof. In some embodiments, the mRNA includes a sequence encoding a T cell receptor or a fragment thereof. In some embodiments, the mRNA includes a sequence that does not encode a B cell receptor or a fragment thereof or a T cell receptor or a fragment thereof.

In some embodiments, the method includes removing the second target nucleic acid, or any other nucleic acid hybridized to the second ligation product, before the second capture sequence hybridizes to the second capture domain of the second capture probe. In some embodiments, the removing includes the use of an RNase. In some embodiments, the RNase includes RNase H. In some embodiments, the removing includes the use of heat.

In some embodiments, the method includes a step of extending a 3′ end of the second ligation product using the second capture probe as a template, thereby generating an extended ligation product; and/or extending the second capture probe using the second ligation product as a template, thereby generating a second extended capture probe.

In some embodiments, the ligation product (e.g., the single-stranded ligation product(s)) hybridizes to the capture domain of the capture probe on the substrate. In some embodiments, the 3′ end of the capture probe is extended using the ligation product as a template. In some embodiments, the 3′ end of the ligation product is extended using the capture probe as a template, thereby generating an extended ligation product. In some embodiments, both the first capture probe, and the extension products hybridized thereto, are extended from their 3′ ends.

In some embodiments, the ligation product is released from the capture probe. In some embodiments, the releasing includes heat. In some embodiments, the releasing includes KOH.

In some embodiments, the released, extended ligation products can be prepared for downstream applications, such as generation of a sequencing library and next-generation sequencing. Generating sequencing libraries are known in the art. For example, the extended ligation products can be purified and collected for downstream amplification steps. The extended amplification products can be amplified using PCR, where primer binding sites flank the spatial barcode and target nucleic acid (e.g., the first target nucleic acid, the second target nucleic acid), or a complement thereof, generating a library associated with a particular spatial barcode. In some embodiments, the library preparation can be quantitated and/or quality controlled to verify the success of the library preparation steps. The library amplicons are sequenced and analyzed to decode spatial information and the target nucleic acid sequence.

In some embodiments, the method includes generating a sequencing library as described herein. In some embodiments, the determining includes sequencing. Methods and systems for sequencing are known in the art and are described herein. In some embodiments, the sequencing is high-throughput sequencing.

### FURTHER STEPS

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for performing the methods as disclosed herein to the biological sample.

In some embodiments, the method includes staining the biological sample. In some embodiments, the staining includes the use of hematoxylin and eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

The biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, the method includes a step of permeabilizing the biological sample (e.g., a tissue section). For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™ Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

In some embodiments, the biological sample is a tissue sample. In some embodiments, the biological sample (e.g., tissue sample) is a tissue microarray. A tissue microarray contains multiple representative tissue samples from different cases assembled on a single histologic slide, and therefore allows high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

In some embodiments, the tissue sample is a fixed tissue sample. For example, fixing the biological sample can include the use of a fixative including: ethanol, methanol, acetone, formaldehyde, paraformaldehyde-Triton, glutaraldehyde, and combinations thereof. In some embodiments, the biological sample can be fixed using PAXgene. For example, the biological sample can be fixed using PAXgene in addition, or alternatively to, a fixative disclosed herein or known in the art (e.g., an alcohol such as ethanol or methanol, acetone, acetone-alcohol, formalin, paraformaldehyde, paraformaldehyde-Triton, glutaraldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which are hereby incorporated by reference in their entirety, for a description and evaluation of PAXgene for tissue fixation.

In some embodiments, the fixed tissue sample is a formalin-fixed paraffin embedded tissue sample, paraformaldehyde fixed tissue sample, a methanol fixed tissue sample, or an acetone fixed tissue sample. In some embodiments, the tissue sample is a fresh frozen tissue sample. In some embodiments, the biological sample is a tissue section. In some embodiments, the biological sample is a fixed tissue section (e.g., a fixed tissue section prepared by any of the methods described herein).

The tissue sample can be obtained from any suitable location in a tissue or organ of a subject, e.g., a human subject. In some embodiments, the tissue sample is a mouse sample. In some embodiments, the tissue sample is a human sample. In some embodiments, the tissue sample can be derived from skin, brain, breast, lung, liver, kidney, prostate, tonsil, thymus, testes, bone, lymph node, ovary, eye, heart, or spleen (e.g., from mouse or human). In some embodiments, the tissue sample is derived from normal or diseased tissue. In some embodiments, the sample is an embryo sample. The embryo sample can be a non-human embryo sample. In some instances, the sample is a mouse embryo sample.

### EXEMPLARY APPLICATIONS

Also provided herein are uses for the disclosed methods. For example, provided herein are methods for determining the presence and/or abundance of an immune cell clonotype at a location in a biological sample. Some embodiments of any of the methods described herein include capturing transcripts to identify an immune cell clonotype. Thus, the disclosed methods of 5′ capture of target nucleic acids in combination with templated ligation can be used for determining the presence and/or abundance of an immune cell clonotype at a location in a biological sample. Identifying the clonal regions, e.g., regions defined by the places where variable (V), diverse (D), and/or joining (J) segments join to from the complementarity determining regions, including CDR1, CDR2, and CDR3, which provide specificity to the TCRs and/or BCRs, and by coupling clonal information to spatial information, it is possible to understand which T-cell and B-cell clonotypes may be specifically interacting with given cell types within a biological sample.

The disclosed methods are also useful in a clinical setting (e.g., monitoring minimal residual disease (MRD)). Patients treated for many cancers often retain an MRD related to the cancer. That is, even though a patient may have by clinical measures a complete remission of the disease in response to treatment, a small fraction of the cancer cells may remain that have escaped destruction. The type and size of this residual population can be an important prognostic factor for the patient's continued treatment. Thus, the more sensitive the measurement of MRD, the more likely that a subsequent course of treatment will be successful.

In particular, a feature of certain acute lymphoblastic leukemias (ALLs) is the sequence evolution of clonotypes associated with the disease. Although treatment outcomes in ALL have improved dramatically over the past fifty years, a significant proportion of patients will ultimately relapse, usually with disease that is highly refractory to additional therapy. It is generally thought that these relapses are due to residual leukemic cells that are resistant to therapy and remain undetected during clinical remission. Clonality of B-cell populations can be assessed by analysis of gene rearrangements that occur at the immunoglobulin heavy chain (IgH) gene locus and the resulting unique VDJ rearrangements are used as clonotypic markers in precursor-B-cell ALL. Precursor-B-cell ALL is generally thought to be a clonal disease resulting from malignant transformation and expansion of a single B-cell; however, changes in clonal IgH rearrangements between initial diagnosis and relapse has been shown in a significant proportion of pre-B ALL cases. Thus, the disclosed methods can be used for diagnosing and monitoring sequence evolution of IgH clonotypes in ALL, particularly precursor B cell ALL.

In many malignant lymphoid and myeloid neoplasms, a diagnostic tissue sample, such as a peripheral blood sample or a bone marrow sample, is obtained before treatment from which a clonotype profile is generated (a diagnostic clonotype profile). One or more disease-correlated clonotypes are identified in the clonotype profile, usually as the clonotypes having the highest frequencies, e.g., greater than 5 percent. After treatment, the presence, absence, or frequency of such correlating clonotypes is assessed periodically to determine whether a remission is holding or whether the neoplasm is returning or relapsing, based on the presence of, or an increase in the frequency of, the correlating clonotypes (or related clonotypes) in a post-treatment clonotype profile. That is, after treatment, minimal residual disease of the cancer is assessed based on the presence, absence, or frequency of the correlating clonotypes and/or related clonotypes, such as clonotypes evolved therefrom by further substitution, or other mechanisms. In one aspect of the invention, a measure of MRD is taken as a frequency of the one or more clonotypes initially identified as being correlated with the cancer together with the clonotypes evolved therefrom after such initial identification.

Provided herein is a method of monitoring minimal residual disease of a B cell leukemia (e.g., acute lymphoblastic leukemia) patient by assaying one or more patient-specific immune cell clonotypes correlated with the B cell leukemia. In some embodiments, the method includes (a) obtaining from the patient a biological sample comprising B-cells; and (b) determining from the biological sample a presence, absence and/or level of one or more patient-specific immune cell clonotypes correlated with the B cell acute lymphoblastic leukemia and/or previously unrecorded clonotypes evolved therefrom. The method can include repeating step (b) one or more times on a successive sample from the patient to monitor the B cell acute lymphoblastic leukemia. In some embodiments, determining the presence, absence and/or level of one or more patient-specific immune cell clonotypes is performed in accordance with any method of determining a location of a target nucleic acid disclosed herein, including methods of 5′ capture of target nucleic acids.

In some aspects, the method includes monitoring and treating a patient. For example, in some embodiments, the methods of monitoring minimal residual disease further include a step of modifying a treatment regimen of the patient based on a presence, absence and/or level of the one or more patient-specific clonotypes and clonotypes related thereto. The patient can be treated in accordance with suitable treatment regimens in the art. After treatment, the presence, absence or frequency of correlating clonotypes is assessed periodically to determine whether a remission is holding or whether the neoplasm is returning or relapsing, based on the presence of, or an increase in the frequency of, the correlating clonotypes (or related clonotypes) in a post-treatment clonotype profile. That is, after treatment, minimal residual disease of the cancer is assessed based on the presence, absence or frequency of the correlating clonotypes and/or related clonotypes. In one aspect of the invention, a measure of MRD is taken as a frequency of the one or more clonotypes initially identified as being correlated with the cancer together with the clonotypes evolved therefrom after such initial identification. If increased numbers of leukemia cells are detected (e.g., between successive MRD measurements), then a relapse has taken place and the treatment regimen is modified to regain a remissive state. The modification may include use of a different chemotherapeutic combination, use of a different administration schedule, use of different amounts of drug, or a switch to a differ kind of therapy.

### Sandwich Configurations

Some steps of the methods described herein can be performed in a biological sample (e.g., in situ) prior to contacting, or bringing in proximity, the biological sample with the array including a plurality of capture probes. In some embodiments, the biological sample is disposed or placed on the array including the plurality of capture probes prior to step (a). In some embodiments, the biological sample is disposed or placed on the array including the plurality of capture probes prior to step (d). In some embodiments, the biological sample is not disposed or placed on the array. For example, the biological sample can be placed on a substrate (e.g., a glass slide) that does not include a spatial array. In some embodiments, the substrate including the biological sample can be aligned with the array (e.g., “sandwiched”) such that at least a portion of the biological sample is aligned with at least a portion of the array. In embodiments where the biological sample is disposed or placed on a substrate, steps (a)-(c) can be performed prior to aligning the substrate with the array as described herein. In embodiments where the biological sample is disposed or placed on a substrate, steps (a)-(c) can be performed after aligning the substrate with the array as described herein.

In some embodiments, one or more extension products generated from the biological sample are released from the biological sample and migrate to a substrate including an array of capture probes where the extension product(s) hybridize to the capture probes of the array. In some embodiments, the release and migration of the extension product(s) to the substrate including the array of capture probes occurs in a manner that preserves the original spatial context of the extension product(s) in the biological sample. In some embodiments, the biological sample is mounted on a first substrate and the substrate including the array of capture probes is a second substrate. In some embodiments, the method is facilitated by a sandwiching process. Sandwiching processes are described in, e.g., U.S. Patent Application Pub. No. 20210189475, WO 2021/252747, and WO 2022/061152A. In some embodiments, the sandwiching process may be facilitated by a device, sample holder, sample handling apparatus, or system described in, e.g., U.S. Patent Application Pub. No. 20210189475, WO 2021/252747, or WO 2022/061152A.

In some embodiments, the sandwiching process includes: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, where the permeabilization agent releases the extension product(s) from the biological sample.

The sandwiching process methods described above can be implemented using a variety of hardware components. For example, the sandwiching process methods can be implemented using a sample holder (also referred to herein as a support device, a sample handling apparatus, and an array alignment device). In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

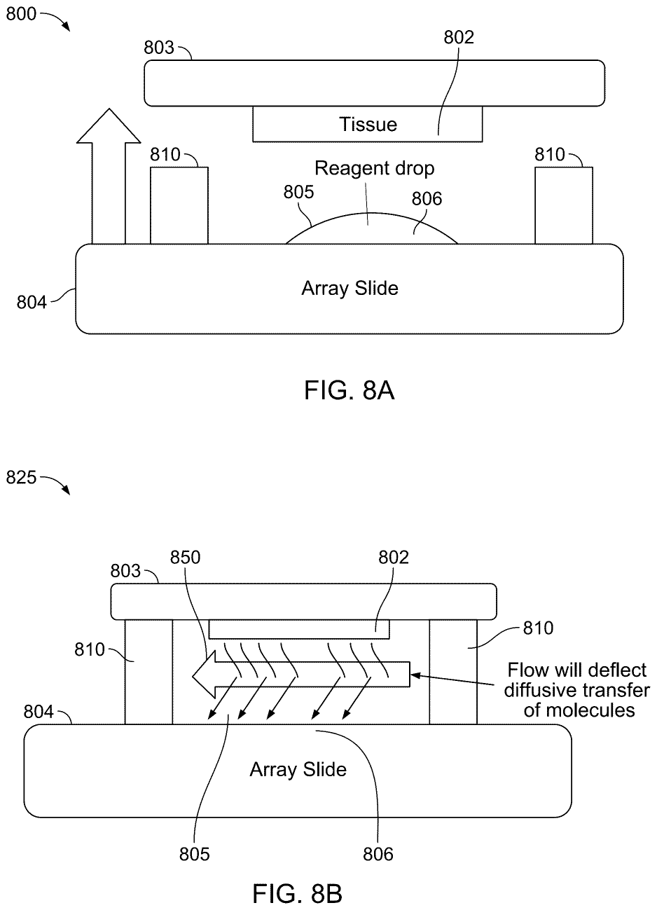

FIG. 8A shows an exemplary sandwiching process 800 where a first substrate (e.g., slide 803), including a biological sample 802 (e.g., a tissue section), and a second substrate (e.g., slide 804 including spatially barcoded capture probes 806) are brought into proximity with one another. As shown in FIG. 8A a liquid reagent drop (e.g., permeabilization solution 805) is introduced on the second substrate in proximity to the capture probes 806 and in between the biological sample 802 and the second substrate (e.g., slide 804 including spatially barcoded capture probes 806). The permeabilization solution 805 can release extension product(s) that can be captured (e.g., hybridized) by the capture probes 806 of the array. As further shown, one or more spacers 810 can be positioned between the first substrate (e.g., slide 803) and the second substrate (e.g., slide 804 including spatially barcoded capture probes 806). The one or more spacers 810 may be configured to maintain a separation distance between the first substrate and the second substrate. While the one or more spacers 810 is shown as disposed on the second substrate, the spacer may additionally or alternatively be disposed on the first substrate.

FIG. 8B shows a fully formed sandwich configuration creating a chamber 850 formed from the one or more spacers 810, the first substrate (e.g., the slide 803), and the second substrate (e.g., the slide 804 including spatially barcoded capture probes 806) in accordance with some example implementations. In FIG. 8B, the liquid reagent (e.g., the permeabilization solution 805) fills the volume of the chamber 850 and can create a permeabilization buffer that allows extension product(s) to diffuse from the biological sample 802 toward the capture probes 806 of the second substrate (e.g., slide 804). In some aspects, flow of the permeabilization buffer may deflect extension product(s) from the biological sample 802 and can affect diffusive transfer of extension product(s) for spatial analysis. A partially or fully sealed chamber 850 resulting from the one or more spacers 810, the first substrate, and the second substrate can reduce or prevent flow from undesirable convective movement of transcripts and/or molecules over the diffusive transfer from the biological sample 802 to the capture probes 806.

### Compositions

Provided herein are compositions including: a) a first probe and a second probe hybridized to a first target nucleic acid and ligated together, where the first probe and the second probe each include a sequence that is substantially complementary to adjacent sequences of the first nucleic acid and where one of the first probe or the second probe includes a first capture sequence; and b) a plurality of primers hybridized to a second target nucleic acid.

Also provided herein are compositions including: a) a first probe and a second probe hybridized to a first target nucleic acid and ligated together, where the first probe and the second probe each include a sequence that is substantially complementary to adjacent sequences of the first nucleic acid and where one of the first probe or the second probe includes a first capture sequence; and b) a plurality of primers hybridized to a second target nucleic acid, where the plurality of primers are extended thereby generating one or more extension products hybridized to the second target nucleic acid.

Also provided herein are compositions including: a) an array including a first plurality of capture probes and a second plurality of capture probes, where a first capture probe of the first plurality of capture probes includes a first spatial barcode and a first capture domain and where a second capture probe of the second plurality of capture probes includes a second spatial barcode and a second capture domain; b) a ligation product hybridized to the first capture domain; and c) one or more extension products hybridized to the second capture domain.

In some embodiments, the composition includes an RNase H enzyme. In some embodiments, the composition includes a ligase. In some embodiments, the first capture probe and/or the second capture probe further include a cleavage domain, one or more functional domains, a unique molecular identifier, and combinations thereof.

In some embodiments, the first and/or the second target nucleic acid is RNA. In some embodiments, the RNA is mRNA. In some embodiments, the mRNA includes a sequence encoding an antibody or a fragment thereof. In some embodiments, the mRNA includes a sequence encoding a T cell receptor or a fragment thereof. In some embodiments, the mRNA includes a sequence encoding a B cell receptor.

In some embodiments, the functional domain includes a primer binding site. In some embodiments, the functional domain includes a sequencing specific site.

In some embodiments, the composition includes a reverse transcriptase. In some embodiments, the composition includes a helicase. In some embodiments, the composition includes one or more single-stranded DNA binding protein selected from the group consisting of: Tth RecA, E. coli RecA, T4 gp32 and ET-SSB. In some embodiments, the composition includes a superhelicase selected from the group consisting of: Rep, PrcA, UvrB, RecBCD, and Tte-Uvrd.

In some embodiments, the extension products further include a sequence complementary to a region of the target nucleic acid that encodes a D sequence of an immune cell receptor.

### Kits

Also provided herein are kits including: (a) an array including a plurality of first capture probes, where a first capture probe of the plurality of capture probes includes: (i) a first spatial barcode; and (ii) a first capture domain that binds a first capture sequence; and a second plurality of capture probes, where a second capture probe of the second plurality of capture probes includes: a second spatial barcode; and a second capture domain; (b) a first primer including a sequence complementary to a target nucleic acid, and a functional domain, where the functional domain includes a primer binding sequence or a sequencing specific sequence; (c) a second primer including a second capture sequence; (d) a plurality of probes including a first probe and a second probe, where the first probe and the second probe are substantially complementary to adjacent sequences of a target nucleic acid, where the second probe includes a first capture sequence that hybridizes the first capture domain; (e) a plurality of enzymes including a ribonuclease and a ligase; and (f) instructions for performing any of the methods described herein.

In some kits, the second primer includes RNA. In some kits, the kit includes a plurality of primers, where the first primer is included in the plurality of primers. In some kits, the kit includes one or more permeabilization reagents. In some kits, the kit includes a reverse transcriptase and/or a polymerase.

In some kits, the first capture probe and/or the second capture probe include one or more additional functional domains, a unique molecular identifier, a cleavage domain, and combinations thereof.

Also provided herein are kits including: (a) an array including a plurality of first capture probes, where a first capture probe of the plurality of capture probes includes: (i) a first spatial barcode; and (ii) a first capture domain that binds a first capture sequence; and a second plurality of capture probes, where a second capture probe of the second plurality of capture probes includes: a second spatial barcode; and a second capture domain; (b) a plurality of primers, where a primer of the plurality of primers includes a sequence complementary to a target nucleic acid and a functional domain, where the functional domain is a primer binding sequence or a sequencing specific site; (c) a plurality of probes including a first probe and a second probe, where the first probe and the second probe are substantially complementary to adjacent sequences of a target nucleic acid, where the second probe includes a first capture sequence that hybridizes to the first capture domain; (e) a plurality of enzymes including a ribonuclease and a ligase; and (f) instructions for performing any of the methods described herein.

In some kits, the first capture probe and/or the second capture probe include one or more additional functional domains, a unique molecular identifier, a cleavage domain, and combinations thereof.

In some kits, the kit further includes a reverse transcriptase. In some kits, the kit includes a polymerase.

In some kits, the first capture domain and/or the second capture domain includes a fixed sequence.

In some kits, the kit includes a helicase. In some kits, the kit includes one or more single-stranded binding proteins includes one or more of: Tth RecA, E. coli RecA, T4 gp32 and ET-SSB.

In some kits, the kit includes a superhelicase, where the superhelicase is selected from the group consisting of: Rep, PrcA, UvrB, RecBCD, and Tte-Uvrd.

### EXAMPLES

### Example 1. 5′ Capture of Target Nucleic Acids

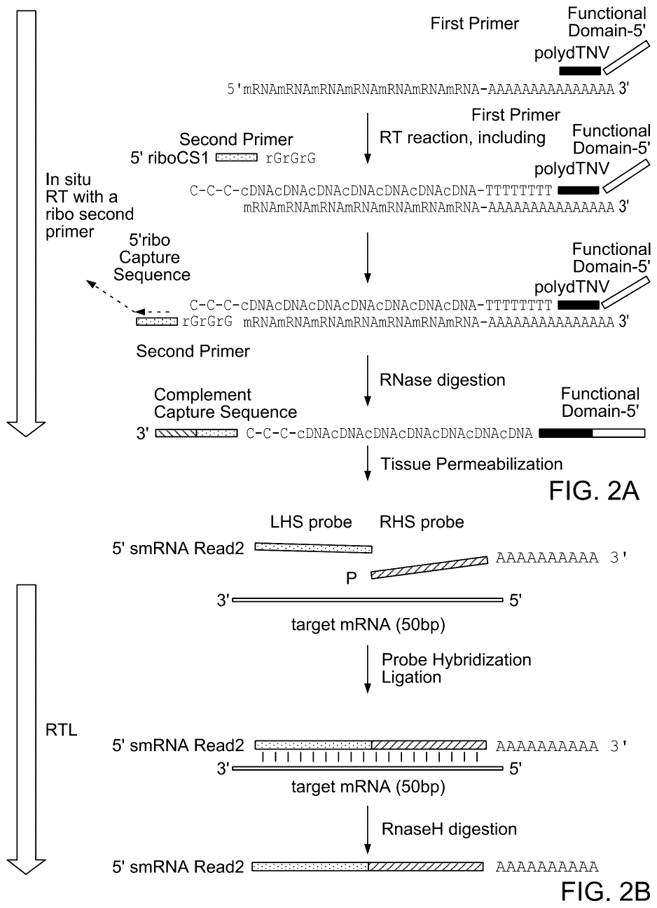

FIG. 2A is a schematic showing generation of a cDNA by in situ reverse transcription of a target nucleic acid (e.g., mRNA) from a first primer including a sequence complementary to the target nucleic acid and a functional domain. The cDNA is further extended using a second primer that includes a capture sequence and a sequence complementary to a non-templated (e.g., homopolynucleotide) sequence.

More specifically, a biological sample containing a target nucleic acid is contacted with a first primer that includes a sequence complementary to the target nucleic acid (e.g., poly(dT) sequence, a poly(dTNV) sequence, a random sequence, a sequence encoding a constant region of a B cell receptor (e.g., an antibody), or a T cell receptor) and a functional domain. In some examples, the functional domain is a primer binding site. In some examples, the functional domain is a sequencing specific site (e.g., Read2 site). The target nucleic acid is reverse transcribed into cDNA and a polynucleotide sequence is added to the 3′ end of the cDNA. FIG. 2A shows a homopolynucleotide sequence comprising cytosines, however, it is appreciated that other polynucleotide sequences can be added to the 3′ end of the cDNA, including a heteropolynucleotide sequence, such as CGC. In some examples, the polynucleotide sequence is added by the reverse transcriptase. In some examples, the polynucleotide sequence is added by a terminal transferase (e.g., terminal deoxynucleotidyl transferase). FIG. 2B is a schematic diagram showing generation of a ligation product in situ by hybridizing a pair of probes to a target nucleic acid. The hybridized probes can be complementary to any portion of the target nucleic acid so long as they are adjacent and can be ligated together to form a ligation product. At least one of the probes of the pair of probes has a capture sequence capable of hybridizing to a capture domain of a capture probe.

A second primer is added where the second primer includes a sequence complementary to the polynucleotide (e.g., a homopolynucleotide sequence, a heteropolynucleotide sequence) sequence added to the 3′ end of the cDNA and a capture sequence. In some examples, the second primer is RNA. After reverse transcription, incorporation of the polynucleotide sequence at the 3′ end of the cDNA, and further extension of the cDNA using the second primer as an extension template, and ligation of the templated probes, an RNase (e.g., RNase H) is contacted with the biological sample (e.g., a tissue section). The RNase degrades the RNA strand of the RNA/cDNA duplex and the RNA strand of the ligation product/RNA duplex, leaving a single-stranded cDNA product (e.g., an extension product) that includes the first primer at its 5′ end and the complement of the capture sequence (e.g., a second capture sequence) capable of hybridizing a capture domain of a capture probe and a single-stranded ligation product including a capture sequence (e.g., a first capture sequence) capable of hybridizing to a sequence in a capture domain of a capture probe.

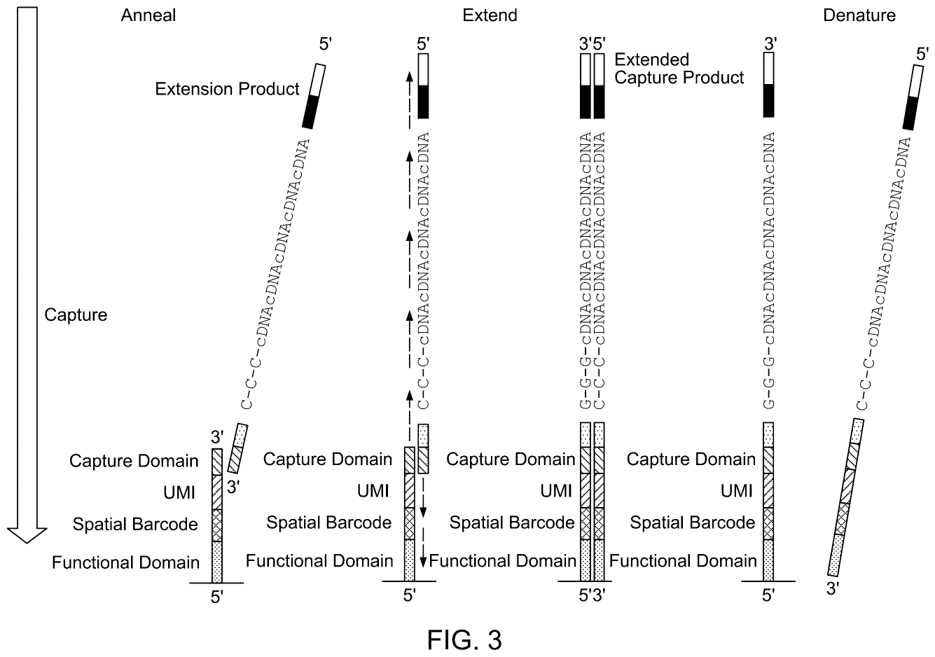

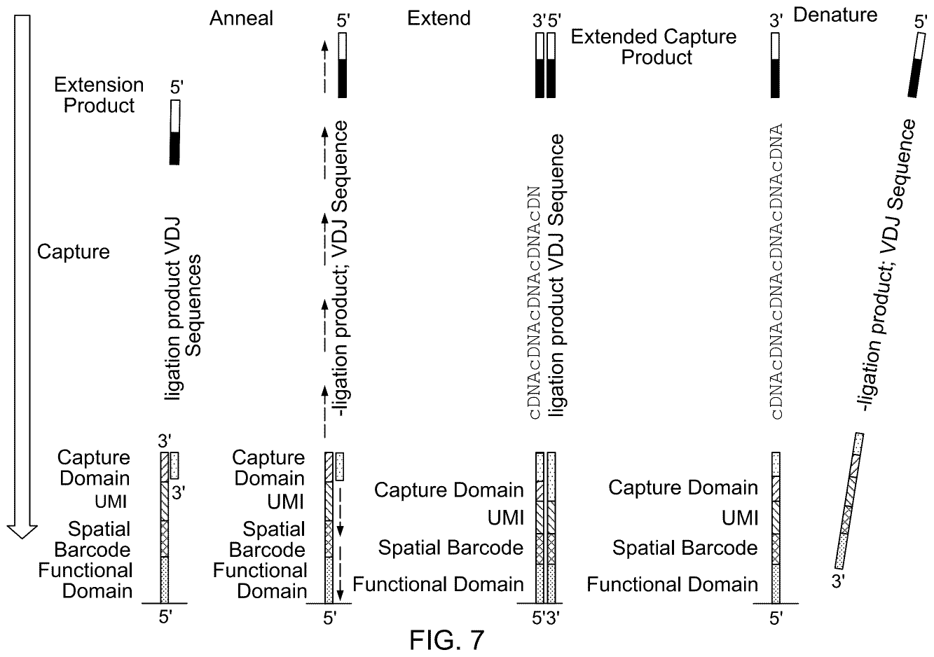

FIG. 3 is a schematic showing capture of the extension product (e.g., the single-stranded cDNA product shown in FIG. 2A) by a capture probe on the substrate. The capture probe is attached to the substrate via its 5′ end and can include one or more functional domains, a spatial barcode, a unique molecular identifier, a capture domain, or a combination thereof. In some examples, the capture probe also includes a cleavage domain. The capture domain hybridizes to the complement of the capture sequence within the extension product (e.g., single-stranded cDNA product) from FIG. 2A. In some examples, the 3′ end of the capture probe is extended using the extension product as a template, thereby generating an extended capture probe. In some examples, the 3′ end of the extension product (e.g., single-stranded cDNA product) is extended using the capture probe as a template thereby generating an extended capture product. In some examples, the 3′ end of the capture probe is extended using the extension product as a template and the 3′ end of the extension product is extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH. FIG. 7 is a schematic diagram showing capture of the ligation product shown in FIG. 2B by a capture probe on the substrate. The capture probe is attached to the substrate via its 5′ end and can include one or more functional domains, a spatial barcode, a unique molecular identifier, a capture domain, or a combination thereof. In some examples, the capture probe also includes a cleavage domain. The capture domain hybridizes to the capture sequence (e.g., the first capture sequence) within the ligation product (e.g., single-stranded ligation product). In some examples, the 3′ end of the capture probe is extended using the extension product as a template, thereby generating an extended capture probe. In some examples, the 3′ end of the ligation product is extended using the capture probe as a template, thereby generating an extended ligation product. In some examples, the 3′ end of the capture probe is extended using the ligation product as a template (e.g., generating an extended capture probe) and the 3′ end of the ligation product is extended using the capture probe as a template (e.g., generating an extended ligation product). In some examples, the extended ligation product is released from the capture probe. In some examples, the extended ligation product is released via heat. In some examples, the extended ligation product is denatured from the capture probe. In some examples, the extended ligation product is denatured from the capture probe with KOH.

FIG. 6 is a schematic diagram showing an embodiment of FIG. 2A where reverse transcription of a target nucleic acid is performed with a plurality of primers. In some examples, reverse transcription is performed using a reverse transcriptase with strand displacement activity. In some examples, reverse transcription is performed with a reverse transcriptase and a helicase. In some examples, reverse transcription is performed with a reverse transcriptase and a superhelicase. In some examples, reverse transcription is performed with one or more single-stranded DNA binding proteins. When a plurality of primers is used to template reverse transcription as shown in FIG. 2A, the resulting extension products can be of different lengths depending on where the primer hybridized to the target nucleic acid (FIG. 6 (bottom)). In some examples, a primer of the plurality of primers can facilitate more than one reverse transcription reaction, thus resulting in two or more extension products generated from the same region of the target nucleic acid hybridized by the primer.

The released, extended captured products can be prepared for downstream applications, such as generation of a sequencing library and next-generation sequencing.

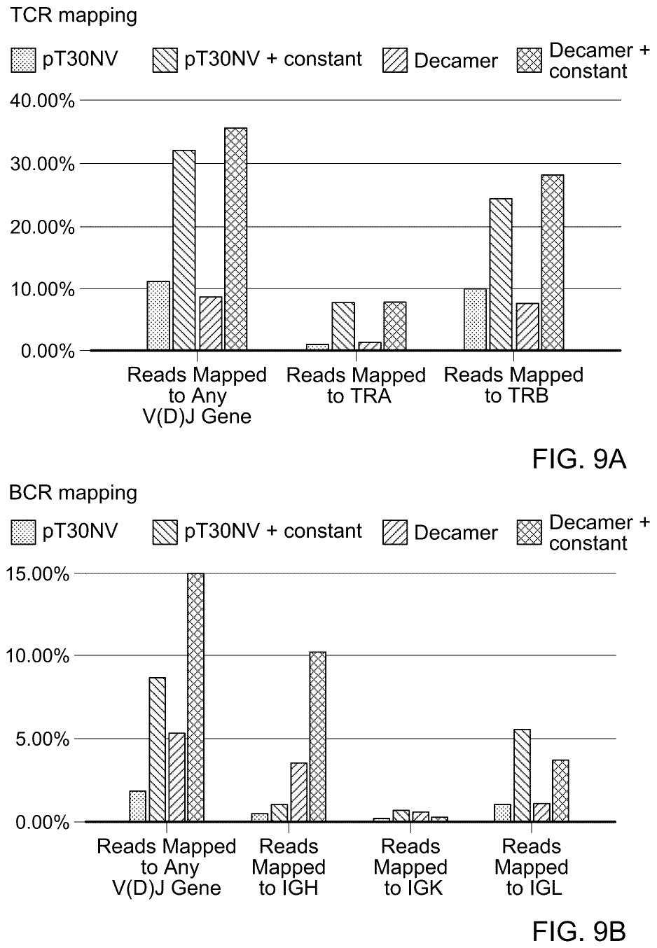

FIGS. 9A-B are graphs showing sequencing mapping rates for T cell receptors (FIG. 9A) and B cell receptors (FIG. 9B) using different primers combinations in a reverse transcription reaction. Briefly, several different primer combinations were tested for their ability to specifically target sequences and prime generation of extension products from nucleic acids encoding T cell receptors and B cell receptors. The plurality of primers used targeted the poly(A) regions of the target nucleic acid and regions encoding the constant region of the B or T cell receptor. This experiment was performed on PBMC FFPE pellets with highly degraded RNA. The data indicates that the TCR and BCR mapping rates improved when further including primers against the constant region. In the absence of RT primers against the constant region of the TCR and BCR, mapping rates were lower.

FIG. 9A shows that a plurality of primers including a poly(dT30NV) sequence only compared with a combination of primers including a plurality of poly(dT30NV) and a plurality of primers to the constant region of T cell receptors results in mapping rates about triple as compared to when only a plurality of primers including a poly(dT30NV) sequence were used. Similarly, FIG. 9A shows that a plurality of primers including a random decamer sequence only compared with a combination of primers including a plurality of random decamer primers and a plurality of primers to the constant region of T cell receptors results in mapping rates about triple as compared to when only a plurality of primers including a random decamer sequence were used. The data show that mapping rates about tripled with respect to reads mapped to any V(D)J gene, reads mapped to a T cell receptor alpha chain, and reads mapped to a T cell receptor beta chain.

FIG. 9B shows that a plurality of primers including a poly(dT30NV) sequence only compared with a combination of primers including a plurality of poly(dT30NV) and a plurality of primers to the constant region of T cell receptors results in mapping rates about triple as compared to when only a plurality of primers including a poly(dT30NV) sequence were used.

Similarly, FIG. 9B shows that a plurality of primers including a random decamer sequence only compared with a combination of primers including a plurality of random decamer primers and a plurality of primers to the constant region of T cell receptors results in mapping rates about triple as compared to when only a plurality of primers including a random decamer sequence were used. The data show that mapping rates about tripled with respect to reads mapped to any V(D)J gene, reads mapped to an immunoglobulin heavy chain, reads mapped to an immunoglobulin kappa light chain, and reads mapped to an immunoglobulin lambda light chain.

Collectively, the data show that 5′ capture of target nucleic acids including V(D)J genes, TCRs, and BCRs is enhanced with a 5′ capture approach as shown in FIG. 2A and a combination of primers including a plurality of p(dTNV30) primers or a plurality of random decamer primers and a plurality of primers to the constant region of nucleic acid target (e.g., TCRs, BCRs, etc.).

### Example 2. 5′ Capture of Target Nucleic Acids

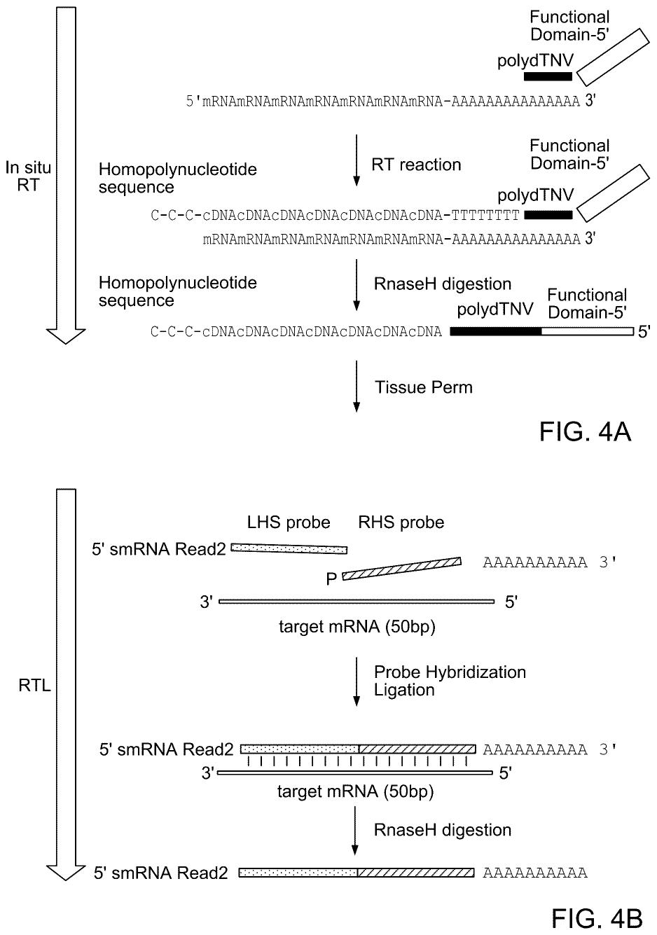

FIG. 4A is a schematic showing generation of a cDNA by in situ reverse transcription of a target nucleic acid (e.g., mRNA) from a primer including a sequence complementary to the target nucleic acid and a functional domain. More specifically, target nucleic acids are contacted with a primer that includes a sequence complementary to the target nucleic acid (e.g., poly(dT) sequence, a poly(dTNV) sequence, a random sequence, a sequence in the target nucleic acid encoding a constant region of a B cell receptor (e.g., an antibody), or a T cell receptor) and a functional domain. In some examples, the functional domain is a primer binding site. In some examples, the functional domain is a sequencing specific site (e.g., Read2 site). The target nucleic acid is reverse transcribed into cDNA and a polynucleotide sequence is added to (or incorporated at) the 3′ end of the cDNA. FIG. 4A shows a homopolynucleotide sequence comprising cytosines, however, it is appreciated that other polynucleotide sequences can be added to the 3′ end of the cDNA, including a heteropolynucleotide sequence, e.g., 5′-CGC-′3. In some examples, the polynucleotide sequence is added by the reverse transcriptase. In some examples, the polynucleotide sequence is added by a terminal transferase (e.g., terminal deoxynucleotidyl transferase).

FIG. 4B is a schematic diagram showing generation of a ligation product in situ by hybridizing a pair of probes to a target nucleic acid. The hybridized probes can be complementary to any portion of the target nucleic acid so long as they are adjacent and can be ligated together to form a ligation product. At least one of the probes of the pair of probes has a capture sequence capable of hybridizing to a capture domain of a capture probe.

After reverse transcription and extension of the 3′ end of the cDNA and hybridization and ligation of the templated probes, an RNase (e.g., RNase H) is contacted with the biological sample (e.g., a tissue section). The RNase degrades the RNA strand of the RNA/cDNA duplex and the RNA strand of the ligation product/RNA duplex, leaving a single-stranded cDNA product (e.g., an extension product) that includes the primer at its 5′ end and the added polynucleotide sequence which is substantially complementary to and capable of hybridizing to a capture domain of a capture probe, and a single-stranded ligation product including a capture sequence capable of hybridizing to a sequence in a capture domain of a capture probe.

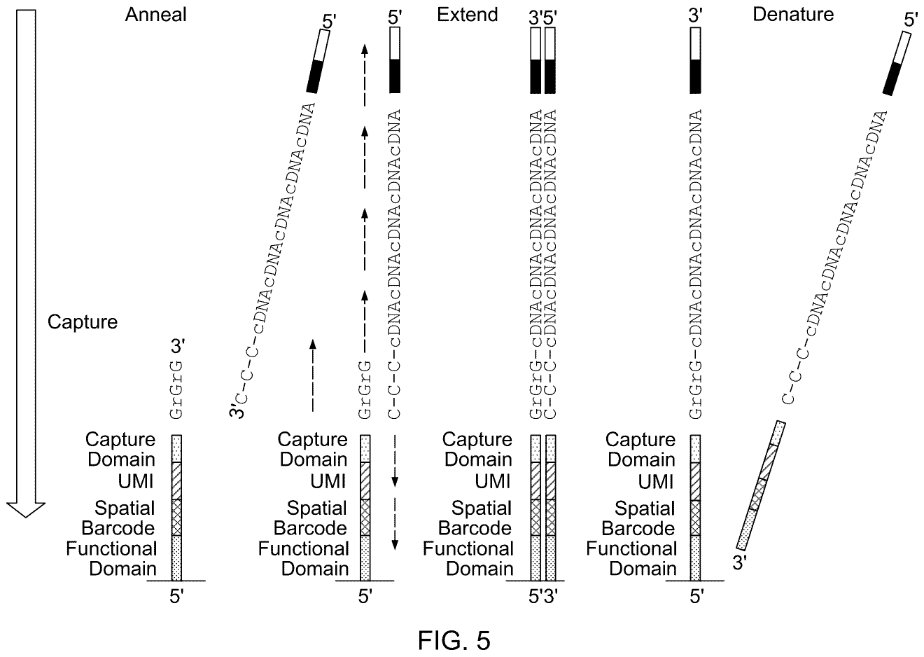

FIG. 5 is a schematic showing capture of the extension product (e.g., the single-stranded cDNA product shown in FIG. 4A by a capture probe on the substrate. The capture probe is attached to the substrate via its 5′ end and can include one or more functional domains, a spatial barcode, a unique molecular identifier, a capture domain, or a combination thereof. In some examples, the capture probe also includes a cleavage domain. The capture domain (which includes RNA in some embodiments) hybridizes to the added polynucleotide sequence which is substantially complementary to and capable of hybridizing to a capture domain of a capture probe within the extension product (e.g., single-stranded cDNA product) from FIG. 4A. In some examples, the 3′ end of the capture probe is extended using the extension product as a template, thereby generating an extended capture probe. In some examples, the 3′ end of the extension product (e.g., single-stranded cDNA product) is extended using the capture probe as a template, thereby generating an extended capture product. In some examples, the 3′ end of the capture probe is extended using the extension product as a template and the 3′ end of the extension product is extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH. FIG. 7 is a schematic diagram showing capture of the ligation product shown in FIG. 4B by a capture probe on the substrate. The capture probe is attached to the substrate via its 5′ end and can include one or more functional domains, a spatial barcode, a unique molecular identifier, a capture domain, or a combination thereof. In some examples, the capture probe also includes a cleavage domain. The capture domain hybridizes to the capture sequence (e.g., the first capture sequence) within the ligation product (e.g., single-stranded ligation product). In some examples, the 3′ end of the capture probe is extended using the extension product as a template. In some examples, the 3′ end of the ligation product is extended using the capture probe as a template thereby generating an extended ligation product. In some examples, the 3′ end of the capture probe is extended using the ligation product as a template and the 3′ end of the ligation product is extended using the capture probe as a template (e.g., generating an extended ligation product). In some examples, the extended ligation product is released from the capture probe. In some examples, the extended ligation product is released via heat. In some examples, the extended ligation product is denatured from the capture probe. In some examples, the extended ligation product is denatured from the capture probe with KOH.

FIG. 6 is a schematic diagram showing an embodiment of FIG. 4A where reverse transcription of a target nucleic acid is performed with a plurality of primers. In some examples, reverse transcription is performed using a reverse transcriptase with strand displacement activity. In some examples, reverse transcription is performed with a reverse transcriptase and a helicase. In some examples, reverse transcription is performed with a reverse transcriptase and a superhelicase. In some examples, reverse transcription is performed with one or more single-stranded DNA binding proteins. When a plurality of primers is used to template reverse transcription as shown in FIG. 4A, the resulting extension products can be of different lengths depending on where the primers hybridized to the target nucleic acid (FIG. 6 (bottom)). In some examples, a primer of the plurality of primers can facilitate more than one reverse transcription reaction, thus resulting in two or more extension products generated from the same region of the target nucleic acid hybridized by the primer.

The released, extended captured products can be prepared for downstream applications, such as generation of a sequencing library and next-generation sequencing.

### Example 3. Capture of Ligation Product Via Templated-Ligation and Gap-Filling

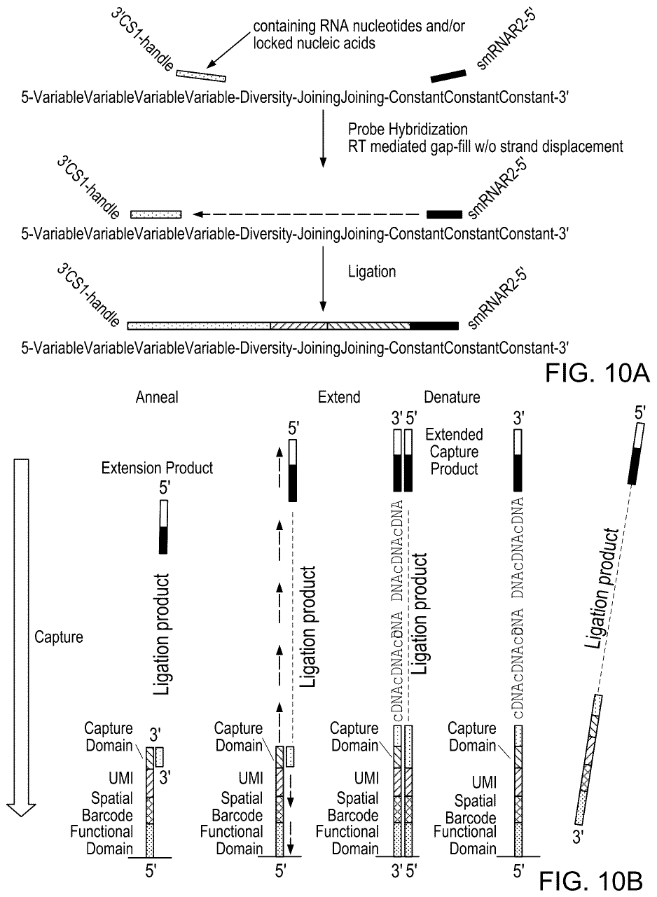

FIG. 10A is a schematic diagram showing generation of a ligation product in situ by hybridizing a pair of probes to a target nucleic acid. The hybridized probes can be complementary to any portion of the target nucleic acid. In some instances, there are one or more intervening nucleotides between the hybridized first probe and the second probe. In some examples, the first probe hybridizes to the constant region of a target nucleic acid. In some examples, the first probe hybridizes to a sequence encoding the constant region of a B cell receptor. In some examples, the first probe hybridizes to a sequence encoding the constant region of a T cell receptor. In some examples, the second probe hybridizes to the variable region of a target nucleic acid. In some examples, the second probe hybridizes to a sequence encoding the variable region of a B cell receptor. In some examples, the second probe hybridizes to a sequence encoding the variable region of a T cell receptor.

The first probe and/or the second probe can include DNA nucleotides, RNA nucleotides, locked nucleic acids, or combinations thereof. In some examples, the first probe includes a portion substantially complementary to the target nucleic acid and a functional domain. In some examples, the functional domain is a primer binding site or a sequencing specific site. In some examples, the second probe includes a portion substantially complementary to the target nucleic acid and a capture sequence capable of binding (e.g., hybridizing) to a sequence in a capture domain of a capture probe.

After the pair of probes hybridize to the target nucleic acid (e.g., DNA, RNA) the intervening nucleic acid sequence between the probe pair functions as a template for an extension (e.g., reverse transcription) reaction. More specifically, a polymerase (e.g., a reverse transcriptase) extends the first probe, thereby generating an extension product (e.g., via a gap-filling reaction). In some examples, the polymerase (e.g., reverse transcriptase) does not possess strand displacement activity. In some examples, the extension product (e.g., the extended first probe) is ligated to the second probe, thereby generating a ligation product. In some examples, the second probe is extended to generate an extension product, which can then be ligated to the first probe to generate the ligation product. In some examples, the target nucleic acid is removed from the ligation product prior to capture of the ligation product by a capture probe on a substrate. In some examples, the target nucleic acid is RNA (e.g., mRNA). In such examples, the RNA can be removed via an endoribonuclease, such as an RNase. In some examples, the RNase is RNase H. In some examples, the target nucleic acid is DNA (e.g., cDNA).

FIG. 10B is a schematic diagram showing capture and extension of the ligation product shown in FIG. 10A by a capture probe on the substrate. The capture probe is attached to the substrate via its 5′ end and can include one or more functional domains, a spatial barcode, a unique molecular identifier, a capture domain, or combinations thereof. In some examples, the capture probe also includes a cleavage domain. The capture domain hybridizes to the capture sequence within the ligation product (e.g., single-stranded ligation product). In some examples, the 3′ end of the capture probe is extended using the ligation product as a template, thereby generating an extended capture probe. In some examples, the 3′ end of the ligation product is extended using the capture probe as a template, thereby generating an extended ligation product. In some examples, the 3′ end of the capture probe is extended using the ligation product as a template and the 3′ end of the ligation product is extended using the capture probe as a template (e.g., generating an extended ligation product). In some examples, the extended ligation product is released from the capture probe. In some examples, the extended ligation product is released via heat. In some examples, the extended ligation product is denatured from the capture probe. In some examples, the extended ligation product is denatured from the capture probe with KOH.

What is claimed is:

1. A method for determining a location of a first target nucleic acid and a second target nucleic acid in a biological sample, the method comprising:
(a) contacting the biological sample with:
(i) a first probe and a second probe, wherein the first probe and the second probe each comprise sequences that are substantially complementary to sequences of the first target nucleic acid, and wherein the second probe further comprises a first capture sequence that is complementary to all or a portion of a first capture domain; and
(ii) a primer comprising a sequence that hybridizes to a complementary sequence in the second target nucleic acid and a functional domain;
(b) hybridizing the first probe and the second probe to the first target nucleic acid;
(c) generating a ligation product by ligating the first probe to the second probe;
(d) hybridizing the first capture sequence of the ligation product to the first capture domain on an array, wherein the array comprises a first plurality of capture probes, wherein a first capture probe of the first plurality of capture probes comprises: a first spatial barcode and the first capture domain;
(e) hybridizing the primer to the second target nucleic acid and extending the primer using the second target nucleic acid as a template to generate an extension product;
(f) incorporating a polynucleotide sequence comprising at least three nucleotides to the 3′ end of the extension product;
(g) hybridizing the polynucleotide sequence of the extension product to a second capture domain on an array, wherein the array comprises a second plurality of capture probes, wherein a second capture probe of the second plurality of capture probes comprises: a second spatial barcode and the second capture domain;
(h) determining (i) the sequence of the first spatial barcode, or a complement thereof, and (ii) all or a portion of the sequence of the ligation product hybridized to the first capture domain, or a complement thereof, and using the determined sequences of (i) and (ii) to determine the location of the first target nucleic acid in the biological sample; and
(i) determining (iii) the sequence of the second spatial barcode, or a complement thereof, and (iv) all or a portion of the sequence of the extension product corresponding to a sequence of the second target nucleic acid, or a complement thereof, and using the determined sequences of (iii) and (iv) to determine the location of the second target nucleic acid in the biological sample.

2. The method of claim 1, wherein the biological sample is disposed on the array comprising the first plurality of capture probes and the second plurality of capture probes.

3. The method of claim 1, wherein the biological sample is disposed on a first substrate, and wherein the array comprising the first plurality of capture probes and the second plurality of capture probes is disposed on a second substrate, wherein the method further comprises aligning the first substrate with the second substrate, such that at least a portion of the biological sample is aligned with at least a portion of the second substrate.

4. The method of claim 1, wherein the hybridizing in (d) and (g) comprise passive migration or active migration of the ligation product and/or the extension product from the biological sample to the array, and optionally, wherein the active migration comprises electrophoresis.

5. The method of claim 1, wherein the polynucleotide sequence in (f) comprises a homopolynucleotide sequence, optionally 5′-CCC-3′; or wherein the polynucleotide sequence in (f) comprises a heteropolynucleotide sequence, optionally 5′-CGC-3′.

6. The method of claim 1, wherein the first capture domain comprises a fixed sequence, optionally poly(T), and wherein the second capture domain comprises a fixed sequence, optionally poly(T).

7. The method of claim 1, wherein the primer comprises a random hexamer, a random decamer, or a homopolymer sequence, optionally wherein the homopolymer sequence comprises a poly(T) sequence.

8. The method of claim 1, wherein the primer comprises:
i) a sequence substantially complementary to a sequence in the second target nucleic acid encoding an antibody;
ii) a sequence substantially complementary to a sequence in the second target nucleic acid encoding a constant region of a B cell receptor; or
iii) a sequence substantially complementary to a sequence in the second target nucleic acid encoding a constant region of a T cell receptor.

9. The method of claim 1, wherein the functional domain comprises a primer binding site or a sequencing specific site.

10. The method of claim 1, wherein the first target nucleic acid is RNA and/or the second target nucleic acid is RNA; optionally wherein the RNA is mRNA.

11. The method of claim 10, wherein the mRNA comprises a sequence encoding a B cell receptor or a fragment thereof or a sequence encoding a T cell receptor or a fragment thereof.

12. The method of claim 1, wherein the first target nucleic acid is DNA.

13. The method of claim 1, wherein the extending in (e) comprises use of a reverse transcriptase.

14. The method of claim 1, wherein incorporating the polynucleotide sequence to the 3′ end of the extension product in (f) comprises use of a reverse transcriptase or a terminal deoxynucleotidyl transferase.

15. The method of claim 1, wherein the method further comprises removing the first target nucleic acid, or any other nucleic acid hybridized to the ligation product, before the first capture sequence hybridizes to the first capture domain of the first capture probe; and/or removing the second target nucleic acid, or any other nucleic acid hybridized to the extension product, before the polynucleotide sequence hybridizes to the second capture domain of the second capture probe; optionally wherein the removing comprises the use of an RNase or heat.

16. The method of claim 1, wherein the method further comprises extending a 3′ end of the extension product of (g) using the second capture probe as a template, thereby generating an extended capture product; and/or extending the second capture probe using the extension product of (g) as a template, thereby generating a second extended capture probe; and/or extending the 3′ end of the ligation product using the first capture probe as a template, thereby generating an extended ligation product; and/or extending the first capture probe using the ligation product as a template, thereby generating a first extended capture probe.

17. The method of claim 1, wherein the first spatial barcode and the second spatial barcode have the same sequence or a different sequence.

18. The method of claim 1, wherein the determining in (h) or (i) comprises sequencing.

19. The method of claim 1, wherein the first probe and the second probe hybridize to sequences that are not adjacent to each other on the first target nucleic acid, wherein the method further comprises extending the first probe with a polymerase, thereby (i) filling in a gap between the first probe and the second probe and (ii) generating an extended first probe.

20. The method of claim 1, wherein the biological sample is a tissue section, wherein the tissue section is a fixed tissue section or a fresh frozen tissue section, optionally wherein the fixed tissue section is a formalin-fixed paraffin embedded tissue section, a paraformaldehyde-fixed tissue section, a methanol-fixed tissue section, or an acetone-fixed tissue section.
